(19) United States
(12) Patent Application Publication
(10) Pub. No.: US 2007/0122800 A1
(43) Pub. Date: May 31, 2007

(54) Methods for quantifying polymer attachment to a particle

(76) Inventors: Seoju Lee, Madison, AL (US); Gary J. Vellekamp, Highland Park, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/603,789

(22) Filed: Nov. 21, 2006

Related U.S. Application Data
(60) Provisional application No. 60/739,739, filed on Nov. 23, 2005.

(51) Int. Cl. C12Q 1/70; C12Q 1/68

(52) U.S. Cl. 435/5; 435/6; 977/924

(57) ABSTRACT

The present invention relates to improved methods for quantifying the degree of polymer attachment of particles with multiple polymer attachment sites. The disclosed methods are useful for gene therapy, particularly gene therapy using pegylated adenoviral vectors.

### REFERENCE TO CROSS RELATED APPLICATIONS

This application claims the benefit of priority under 35 USC 119(e) of provisional patent application U.S. Ser. No. 60/739,739 filed Nov. 23, 2005, the disclosure of which is hereby incorporated by reference in its entirety.

### FIELD OF THE INVENTION

The present invention relates to methods for quantifying the degree of polymer attachment of particles having multiple polymer attachment sites. The disclosed methods are useful for gene therapy, particularly gene therapy using pegylated adenoviral vectors.

### BACKGROUND OF THE INVENTION

As with certain successful protein therapeutics, the attachment of polymers such as polyethylene glycol (PEG) molecules to larger particles such as viral particles has been studied to augment their potential for various therapies (Francis et al., Int. J. Hematol. 68:1-18 (1998); O'Riordan et al., Hum. Gene Ther. 10:1349-1358 (1999)). For example, pegylation of recombinant adenovirus (rAd) has been reported to reduce innate immunity and neutralizing antibodies, improve tissue retargeting, and extend gene expression (O'Riordan et al., Hum. Gene Ther. 10:1349-1358 (1999)). Evaluation of these results, both in vitro and in vivo, provides examples of pegylated rAds that retain their ability to transduce cells and tissues, show reduced cytotoxic T-cell production, and extend the time of gene expression. These pegylated rAds are also protected from antibody neutralization, and allow expression after administration to animals previously immunized with unmodified virus. Evidence of increased stability on storage or in gastric or pancreatic fluids was also observed (Croyle et al., Hum. Gene Ther. 11:1713-1722 (2000); Chen et al., Gene Therapy 10:991-998 (2003)). A recent report of an rAd that was conjugated with PEG linked to an RGD peptide or an E-selectin-specific antibody showed both the elimination of its natural tropism for coxsackie-adenovirus receptor-positive cells and its retargeting to activated endothelial cells (Ogawara et al., Hum. Gene Ther. 14:433-443 (2004)).

Unlike proteins, rAd particles have thousands of potential target sites for PEG conjugation. The reliable measurement of the average number of PEG molecules attached to each virion, or the degree of pegylation (DP), is crucial not only for the characterization of pegylated virus vectors but also for the understanding and optimization of a pegylation reaction itself.

Two methods for determining the amount (and stability) of PEG conjugated per particle of adenovirus prepared for in vivo studies have been reported previously. One method used a biotin-labeled PEG linker to modify the surface of rAd vectors. Following disruption of the modified vectors, ELISA analysis was applied to quantify the biotin-labeled pegylated viral proteins with avidin-horseradish peroxidase (O'Riordan et al. Hum. Gene Ther. 10:1349-1358 (1999)). The other method treated the pegylated virus with fluorescamine to determine the amount of PEG-blocked lysine groups relative to unpegylated controls (Croyle et al., Hum. Gene Ther. 11:1713-1722 (2000)). Both techniques were thought to be problematic. In particular, the results of the previously known disruptive or indirect methods do not reveal the full complexity of the potential variables of the pegylation reactions. They do not consider that since each particle in a rAd target population has multiple, varied pegylation sites, the sequential alteration of each site affects the quality of the subsequent reaction and the resulting conjugate. Also not factored in by these methods are the effects of the purification conditions, the change of surface properties and stabilities of this new pegylated rAd population distribution. For instance, it is not clear as to even how many linear PEG molecules are conjugated per particle in these preparations.

In view of the clear therapeutic advantages inherent in developing polymer conjugated forms of larger particles such as virus particles, there is a need for simple, sensitive, non-disruptive assays to reliably characterize preparations of polymer-particle conjugates. The present invention addresses these needs by providing a method for better characterizing the properties of polymer-conjugated forms of large particles such as viruses.

### SUMMARY OF THE INVENTION

The present invention provides a method for determining the average degree of polymer attachment of a polymer-particle conjugate preparation comprising the steps of (a) measuring the density of a polymer-particle conjugate preparation having an unknown average degree of polymer attachment; (b) measuring the density of a polymer-particle conjugate preparation having a known average degree of polymer attachment; and (c) comparing the density of the polymer-particle conjugate preparation having the known average degree of polymer attachment versus the density of the polymer-particle preparation having the unknown average degree of polymer attachment. In preferred embodiments of the invention, density is measured by analytical ultracentrifugation.

In one embodiment of the invention, the polymer-particle conjugate preparations are pegylated recombinant adenovirus (PEG-rAd) preparations, and the method for measuring the density of the preparations is by analytical ultracentrifugation (AUC) on CsCl gradients. In a particular embodiment, the PEG-rAd preparation having the known average degree of pegylation is a fluorescein-labeled PEG-rAd preparation, and the average degree of pegylation of the fluorescein-labeled PEG-rAd preparation is determined by size exclusion (SE) HPLC with fluorescence quantification of the virus peak.

### BRIEF DESCRIPTION OF THE FIGURES

FIG. 1 shows the chemical structure of a fluorescein-labeled PEG-SPA linker.

FIG. 2 depicts purification of fluoro-PEG-rAd by size exclusion chromatography on Superdex 200 H/R. Absorbance was monitored at 260 nm. 2A shows a chromatography absorbance profile. Preparative chromatography: 1.0 ml of final pegylation reaction (1% (w/v) linker concentration) was loaded on 1×30 cm column equilibrated and run in 14 mM Tris, 11 mM sodium phosphate, 2 mM MgCl₂, 2% sucrose, 10% (w/v) glycerol, pH 8.1 at 4° C. (Buffer A). The first peak is the pegylated rAd eluting at the column void volume. The second peak contains potentially three PEG-related molecules: fluoro-PEG from hydrolysis, fluoro-PEG-Tris, and any trace unreacted fluoro-PEG-SPA. The third peak is NHS. 2B shows a chromatogram depicting analytical chromatography: the virus peak eluting at approximately 16 minutes in the preparative chromatography above was pooled (pool concentration was 0.46×10¹² particle/ml) and a 100 μl injection was made on the same column. The arrows are the elution positions of pegylated rAd (1); fluoro-PEG and fluoro-PEG-Tris (2); and N-hydroxy succinimide (NHS)(3).

FIG. 3 provides graphs depicting fluorescent size exclusion chromatography of fluoro-PEG-SPA and fluoro-PEG-rAd. 3A show the fluorescence profile of 30 μl of freshly-prepared (darker line) or aged (˜10 days at 4° C.) (lighter line) fluoro-PEG-SPA linker at a concentration of 2.65×10¹³ molecules/ml. 3B shows the standard curve of the fluorescence peak area of the freshly-prepared fluoroPEG-SPA at varying injection volumes on the Superdex 200 HR column. 3C is the fluorescence profile of 50 μl injection of fluoro-PEG-rAd produced at 1.0% (w/v) linker concentration. The virus sample concentration was 0.347×10¹² particle/ml before a 20-fold dilution with the fluorescence SE-HPLC buffer prior to injection. 3D shows the standard curve of the fluorescence peak area of fluoro-PEG-rAd at varying injection volumes on the Superdex 200 HR column.

FIG. 4 is a graph depicting the effects of pegylation reaction conditions on the degree of pegylation of fluoro-PEG-rAd. Reactions were performed at the indicated percent fluoro-PEG-SPA concentration and the resulting purified pegylated rAds were analyzed by fluorescent SE HPLC to determine the degree of pegylation. 4A shows the effect of virus concentration. Pegylation reactions were performed at 0.55×10¹² particles/ml (open circles) or 0.91×10¹² particles/ml (solid triangles). 4B depicts the effect of pH. Reactions were performed at an initial pH of 8.3 (open circles) or 9.0 (solid triangles) both at a virus concentration of ˜0.9×10¹² particles/ml.

FIG. 5 is a graph depicting the effect of Tris buffer concentration on the degree of pegylation on of fluoro-PEGrAd. Reactions were performed at the indicated Tris concentration and the resulting purified pegylated rAds were analyzed by fluorescent SE HPLC to determine the degree of pegylation. The fluoro-PEG-SPA concentration was 2%, the rAd concentration was 0.5×10¹² particles/ml and the initial pH was ˜8.2.

FIG. 6 provides anion exchange chromatography analysis of pegylated rAds. 6A shows Resource Q HPLC retention time of the purified PEG-rAd prepared at differing % linker concentrations. p53-rAd pegylated with varying fluoro-PEG-SPA linker concentrations at 5.5 (open diamonds) or 9.1 (open squares)×10¹¹ virus particles/ml (solid line); β-gal rAd pegylated with varying PEG-SPA linker concentrations at 5.5×10¹¹ particle/ml (open circles; dashed line). Arrows indicate estimated DP at the indicated % linker concentration. 6B shows resource Q HPLC peak width (ratio of peak height to peak area) of the purified PEG-rAd prepared at differing % linker concentrations. p53-rAd pegylated with varying fluoro-PEG-SPA linker concentrations at 5.5 (open diamond) or 9.1 (open square)×10¹¹ virus particles/ml.

FIG. 7 depicts SDS-PAGE analysis of pegylated rAds. Samples of fluoro-PEG-rAd and PEG-rAd produced at varying linker concentrations as indicated were run on SDS-PAGE. The gel was stained with Coomassie blue (7A) or imaged for fluorescence (7B). Arrows indicate the migration positions of the observed adenovirus proteins. In 7C, the normalized hexon band intensity of the pegylated rAds from the Coomassie blue stained gel was determined by densitometry scanning and plotted versus the % linker concentration used in the pegylation reaction. The symbols are either fluoro-PEG-rAd (open diamond) or PEG-rAd (open square).

FIG. 8 provides analytical ultracentrifugation in CsCl density gradients of pegylated rAds. CsCl was added to a mixture of: A. PEG-rAds produced at 0%, 1%, 4%, or 8% linker concentration; B. fluoro-PEG-rAds produced at 0%, 1.0%, 2.8%, 4.9%, 7.4%, and 10.4% linker concentration; or C. the pegylated rAd samples in B above (at a one-third lower rAd concentration) plus the PEG-rAd produced at 4% linker concentration as in A above. These samples were run on the analytical ultracentrifuge. The profiles of UV absorbance at 260, 280, and 320 nm versus the centrifugation radius are displayed after 16 hours at 30,000 RPM.

FIG. 9 is a graph depicting the stability at 4° C. of pegylated rAds. Samples of fluoro-PEG-rAd prepared at the indicated linker concentrations were incubated at 4° C. for various times and then analyzed with fluorescent SE HPLC. The plot shows the percent change in the fluorescence of the rAd peak position versus the incubation time.

FIG. 10 is a graph depicting the stability after multiple freeze/thaw cycles of pegylated rAds. Samples of fluoro-PEG-rAd prepared at the indicated linker concentrations were subjected to multiple cycles of freezing at −80° C. and thawing at 25° C., and then analyzed with fluorescent SE HPLC. The plot shows the percent change in the fluorescence of the rAd peak position versus the number of freeze/thaw cycles.

FIG. 11 provides fluorescence profiles on size exclusion chromatography of pegylated rAds after multiple freeze/thaw cycles. The fluoro-PEG-rAd prepared at a 1.0% linker concentration was subjected to multiple freeze/thaw cycles as in FIG. 10 and analyzed by fluorescent SE HPLC. The florescence profiles are shown for this vector after 1 (A), 6 (B), or 14 (C) freeze/thaw cycles. The arrows indicate the peak elution positions.

### DETAILED DESCRIPTION OF THE INVENTION

The invention disclosed herein provides a method for determining the degree of polymer attachment of a polymer-particle conjugate preparation. The invention is based, in part, on a discovery by the inventors that the addition of multiple polyethylene glycol (PEG) molecules (via specific pegylation reaction) to a preparation of adenoviral vectors (rAd) has a negative effect on the density of the rAd preparation as determined by analytical ultracentrifugation (AUC) on CsCl gradients. This ability to obtain a reproducible and precise measure of density can be used to reliably determine the average degree of pegylation (DP) of a preparation of pegylated rAds relative to a chosen standard.

In the examples provided below, the DP of an rAd preparation was determined relative to the DP of a preparation of flourescein-labeled PEG-rAds as determined by size exclusion (SE) HPLC with fluorescence quantification of the virus peak (Example 5). Other standards can also used, as will be discussed below. Examples 4 and 5 describe how, using a PEG monomer with a fluorescein-label at the chain terminal, a fluorescent size exclusion HPLC assay was developed that accurately and reproducibly determined the average number of PEG molecules per rAd particle, i.e., the degree of pegylation (DP). This assay was then used to evaluate the effect of reaction variables on the degree of pegylation and the stability of the purified pegylated vectors (Examples 13 and 14).

Once the DP of the fluorescein-labeled preparation was determined, a set of these purified samples prepared at differing linker concentrations was compared to a set of previously prepared, non-fluorescent PEG-rAds having an unknown average degree of pegylation using analytical ultracentrifugation (AUC) (Example 11). The results obtained with AUC were confirmed with a variety of non-fluorescent analytical techniques, including SDS-PAGE (Example 8), anion exchange chromatography (Example 9), and RP-HPLC (Example 10). The DP of the non-fluorescent sample prepared at 4% linker concentration was determined by AUC to have a DP of approximately 1800 by comparison to the fluoro-PEG-rAd standards run together with it on AUC. The ability to quickly and reproducibly determine the relative average DP of a pegylated adenovirus preparation by the claimed AUC methods is therefore a significant advance that will be useful in ongoing gene therapy research.

The methods disclosed herein are equally useful to determine the degree of conjugation of other polymers to other particles having multiple polymer attachment sites in a population of conjugates. In this regard, the term “polymer-particle conjugate” as used herein is defined as an entity comprising a particle having multiple polymer attachment sites, to which one or more polymers has been conjugated. Examples of such polymer-particle conjugates are abundant in the art.

In addition to the pegylated adenoviruses exemplified herein, the polymer-particle conjugates referred to in the invention may consist of other particles having multiple polymer attachment sites. Representative particle within this definition include but are not limited to other viruses, oligonucleotide or oligonucleotide complexes (Vinogradov et al., Bioconjug. Chem 10:851-860 (1999); Bartsch et al., Mol. Pharmacol. 67:883-890 (2005)), fullerenes, dendrasomes, nanoparticles (Nobs et al., Eur. J. Pharm. Biopharm. 58:483-490 (2004); Passirani et al., Pharm. Res. 15:1046-1050 (1998)), nanocapsules (Mosqueira et al., Biomaterials 22:2967-2979 (2001)), microparticles or microgels (Alakhov et al., J. Pharm. Pharmacol. 56:1233-1241 (2004)). A preferred subset of particles containing multiple polymer attachment sites according to the invention are those which comprise a sequence of nucleic acids. Since particles containing nucleic acid sequences tend to have a higher density than many non-nucleic acid particles, such particles are well suited for characterization according to the claimed methods. Thus, when determining the relative average degree of polymer attachment of a polymer-particle conjugate preparation wherein the particle comprises a sequence of nucleic acids, a lower density would be expected to indicate a higher degree of polymer attachment. Representative particles which contain nucleic acid sequences include but are not limited to viral or non-viral DNA, RNA, or synthetic nucleic acid sequences. Preferred viral vectors are adenoviral vectors. Preferred non-viral vectors include oligonucleotides and oligonucleotide complexes (Vinogradov et al., Bioconjug. Chem 10:851-860 (1999); Bartsch et al., Mol. Pharmacol. 67:883-890 (2005)).

Polymers of the polymer-particle conjugate that may be measured by the claimed methods include but are not limited to polyethylene glycol and other synthetic polymers, proteins (see, e.g., Nobs et al., Eur. J. Pharm. Biopharm. 58:483-490 (2004); Jia et al., Biotech. Bioeng. 84:406-414 (2003)), oligonucleotides (Farokhzad et al., Cancer Res. 64:7668-7672 (2004); Douglas et al., J. Biomater. Sci. Polym. Ed. 16:43-56 (2005)), oligosaccarides (Passirani et al., Pharm. Res. 15:1046-1050 (1998)), lipids and detergents (Alakhov et al., J. Pharm. Pharmacol. 56:1233-1241 (2004)). The polymers can be conjugated to a particle by methods known in the art. By way of example, and not limitation, the polymer can be conjugated covalently, (see, e.g, U.S. Pat. Nos. 5,711,944 and 5,951,974) or non-covalently (see, e.g., WO2005/012407).

As will be recognized by one skilled in the art, the conditions of the claimed AUC methods may be optimized for the particular polymer-particle conjugate being measured. Specifically, the potential resolution between polymer-particle conjugates of differing density and the stability of the polymer-particle conjugates during the AUC analysis may be improved by varying the solution composition (e.g., CsCl or glycerol concentration, pH, etc.) and the centrifugation speed and time. Likewise, the relative densities of the polymer and particle components of the polymer-particle conjugates must be considered when considering the relevance of a higher or lower density reading. For example, in the exemplified embodiment, the PEG molecules have a lower density than the rAd particles, such that consecutive addition of PEG molecules to the rAd particle results in a lower overall density reading. One skilled in the art would be able to factor the relative densities of other polymers and other particles to assess the relevance of the density readings.

Analytical Centrifugation (AUC)

The analytical ultracentrifuge is commercially available through Beckman Coulter, Inc., Fullerton Calif. Analytical centrifugation in CsCl gradients is a well known method, used primarily to characterize proteins (Burlingham et al., Virology 60:419-430 (1974)). A primary advantage of the AUC technique is that separation of materials with differing densities is monitored during the centrifugation in situ and in real time without disturbing the details of the absorbance profile. Although velocity centrifugation using AUC has been applied extensively for protein size characterization (Schuck, Anal. Biochem 272:199-208 (1999); Lebowitz et al., Protein Sci. 11:2067-2079 (2002)), this equilibrium centrifugation technique using AUC to observe adenovirus particle heterogeneity is rarely reported (Burlingham et al., Virology 60:419-430 (1974)).

By way of example, and not limitation, the analytical ultracentrifugation can be performed on a density gradient comprising one or more of the following Cesium Chloride, Glycerol, Sucrose, Rubidium Chloride, other density modifying agents or combinations thereof. In one embodiment Cesium Chloride in combination with glycerol is used to form the density gradient. Examples of ranges of Cesium Chloride and glycerol that may be used include, but are not limited to, between about 415 mg Cesium Chloride to about 519 mg Cesium Chloride per ml of the buffer solution comprising the PEG-rAd sample and between about 2% to about 40% glycerol (w/v), more preferably between about 5% to about 10% glycerol (w/v). The buffer solution may further comprise up to about 10% sucrose, more preferably between about 1% to about 5% sucrose. By way of example, and not limitation, ranges of Cesium Chloride between about 415 mg Cesium Chloride to about 519 mg Cesium Chloride per ml of the buffer solution comprising the PEG-rAd sample and about 10% glycerol (w/v) and about 2% sucrose yield density gradients of between about 1.30 g/ml and about 1.34 g/ml. The slope of the density gradient can also be varied by altering the centrifugation speed. By way of example, and not limitation, for a PEG-rAd sample, the centrifugation speed may be between about 20,000 to about 45,000 rpm.

In a particular embodiment, about 456 mg of Cesium Chloride per ml of the buffer solution comprising the PEG-rAd sample, in about 10% glycerol (w/v) and about 2% sucrose can be used to yield a density of about 1.32 g/ml.

Other Standards

In the examples provided below, the DP of a pegylated rAd preparation was determined relative to the DP of a preparation of flourescein-labeled PEG-rAds as determined by size exclusion (SE) HPLC with fluorescence quantification of the virus peak (Example 7). Other methods which are known in the art can also used to determine the relative degree of pegylation of the polymer-particle conjugate preparation to be used as a standard. For example, the prior art method of using a biotin-labeled PEG linker that had its DP determined by ELISA analysis of the biotin-labeled pegylated rAd with avidin-horseradish peroxidase (O'Riordan et al., Hum. Gene Ther. 10:1349-1358 (1999)) can be used as a standard. Alternately, the pegylated virus preparation can be treated with fluorescamine to quantify the loss of lysine groups relative to unpegylated controls (Croyle et al., Hum. Gene Ther. 11:1713-1722) for determination of the average degree of pegylation of the standard preparation. Also envisioned in the methods of the present invention is the use of a standard in which the degree of pegylation of the PEG-rAd preparation is defined by the percent linker concentration used in the pegylation reaction. Additionally, relative standards may be used. For example, unknown samples may be quantitatively compared to a standard material prepared by a defined process.

Recombinant Adenoviruses (rAd)

Recombinant Adenoviruses (rAd) are widely used to deliver genes into cells for vaccines or gene therapy (Alemany et al., J. Gen. Virol. 81(11):2605-2609 (2000); Vorberger & Hunt, Oncologist 7(1):46-59 (2002); Mizuguchi & Hayakawa, Hum. Gene Ther. 15(11):1034-1044 (2004); Basak et al., Viral Immunol. 172:182-96 (2004).

The term “recombinant” refers to a genome which has been modified through conventional recombinant DNA techniques.

The term “virus” as used herein includes not only naturally occurring viruses but also recombinant viruses, attenuated viruses, vaccine strains, and so on. Recombinant viruses include, but are not limited to, viral vectors comprising a heterologous gene. The term recombinant virus includes chimeric (or even multimeric) viruses, i.e. vectors constructed using complementary coding sequences from more that one viral subtype. See, e.g., Feng et al. Nature Biotechnology 15:866-870 (1997). In some embodiments, helper function(s) for replication of the viruses is provided by the host cell, a helper virus, or a helper plasmid. Representative vectors include, but are not limited to, those that will infect mammalian cells, especially human cells, and may be derived from viruses such as retroviruses, adenoviruses, adeno-associated viruses, herpesviruses, and avipox viruses.

In one embodiment, the virus is adenovirus. The term “adenovirus” is synonymous with the term “adenoviral vector” and refers to viruses of the genus adenoviridae. The term “recombinant adenovirus” is synonymous with the term “recombinant adenoviral vector” and refers to viruses of the genus adenoviridiae capable of infecting a cell, whose viral genomes have been modified through conventional recombinant DNA techniques. The term recombinant adenovirus also includes chimeric (or even multimeric) vectors, i.e. vectors constructed using complementary coding sequences from more than one viral subtype.

The term adenoviridae refers collectively to animal adenoviruses of the genus mastadenovirus including but not limited to human, bovine, ovine, equine, canine, porcine, murine and simian adenovirus subgenera. In particular, human adenoviruses include the A-F subgenera as well as the individual serotypes thereof. For example, any of adenovirus types 1, 2, 3, 4, 4a, 5, 6, 7, 7a, 7d, 8, 9, 10, 11 (Ad11A and Ad11P), 12, 13, 14, 15, 16, 17, 18, 19, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91 may be produced in a cell culture of the invention. In the preferred practice of the invention, the adenovirus is or is derived from the human adenovirus serotypes 2 or 5.

In one embodiment of the invention, the adenovirus comprises a wild-type, unmutated genome. In another embodiment, the adenovirus is a recombinant adenovirus or a recombinant adenoviral vector, which comprises a mutated genome; for example the mutated genome may be lacking a segment or may include one or more additional, heterologous gene. In one embodiment, the recombinant adenoviral vector is the adenoviral vector delivery system which has a deletion of the protein IX gene (see International Patent Application WO 95/11984, which is herein incorporated by reference).

In another embodiment, the adenovirus is a selectively replicating recombinant adenovirus or a conditionally replicating adenovirus, i.e., an adenovirus that is attenuated in normal cells while maintaining virus replication in tumor cells, see, e.g., Kirn, D. et al., Nat. Med. 7:781-787 (2001); Alemany, R. et al. Nature Biotechnology 18: 723-727 (2000); Ramachandra, M. et al., Replicating Adenoviral Vectors for Cancer Therapy in Pharmaceutical Delivery Systems, Marcel Dekker Inc., New York, pp. 321-343 (2003).

In one embodiment of the invention, the selectively replicating recombinant adenovirus or the adenoviral vector is such as those described in published international application numbers, WO 00/22136 and WO 00/22137; Ramachandra, M. et al., Nature Biotechnol. 19: 1035-1041 (2001); Howe et al., Mol. Ther. 2(5):485-95 (2000); and Demers, G. et al. Cancer Research 63: 4003-4008 (2003).

A selectively replicating recombinant adenovirus may also be described as, but not limited to, an “oncolytic adenovirus”, an “oncolytic replicating adenovirus”, a “replicating adenoviral vector”, a “conditionally replicating adenoviral vector” or a “CRAV”.

In another embodiment of the invention, the adenovirus is 01/PEME, also known as cK9TB or K9TB, that is modified to attenuate replication in normal cells by deletions in the E1a gene and the E3 region, insertion of a p53 responsive promoter driving an E2F antagonist, E2F-Rb, and insertion of a major later promoter regulated E3-11.6K gene and is described, for example, in Ramachandra et al., Nature Biotechnol. 19: 1035-1041 (2001); United States Patent Application Publication Number US2002/0150557; and Demers et al. Cancer Research 63: 4003-4008 (2003).

As used herein the terms, “rAd production cell”, “producer cell”, and “packaging cell” are synonyms and mean a cell able to propagate recombinant adenoviruses by supplying a product required for efficient viral growth. A variety of mammalian cell lines are publicly available for the culture of recombinant adenoviruses. For example, the 293 cell line (Graham & Smiley, J. Gen Virol. 36:59-72 (1977)) has been engineered to complement the deficientcies of E1 function.

The rAd production cells or cell lines may be propagated using standard cell culture techniques (see e.g., R. I. Freshney, Culture of Animal Cells—A Manual of Basic Techniques, Second Edition, Wiley-Liss, Inc. New York, N.Y., 1987).

Cells may grow in serum-containing or serum-free conditions. The suspension culture may be shaken, rocked, agitated, rolled or stirred to maintain the cells in suspension.

“A549” is a lung carcinoma cell line which is commonly known in the art. In one embodiment, the A549 cell is ATCC strain CCL-185.

The recombinant adenovirus production cells or production cell line may be propagated or grown by any method known in the art for mammalian cell culture. Propagation may be done by a single step or a multiple step procedure. For example, in a single step propagation procedure, the production cells are removed from storage and inoculated directly to a culture vessel where production of virus is going to take place. In a multiple step propagation procedure, the production cells are removed from storage and propagated through a number of culture vessels of gradually increasing size until reaching the final culture vessel where the production is going to take place. During the propagation steps, the cells are grown under conditions that are optimized for growth. Culture conditions, such as temperature, pH, dissolved oxygen level and the like are those known to be optimal for the particular cell line and will be apparent to the skilled person or artisan within this field (see e.g., Animal Cell culture: A Practical Approach 2^{nd} edition, Rickwood, D. and Hames, B. D. eds., Oxford University Press, New York (1992)).

The rAd production cells or rAd production cell lines may be grown and the rAd production cells or rAd production cells producing virus may be cultured in any suitable vessel which is known in the art. For example, cells may be grown and the infected cells may be cultured in a biogenerator or a bioreactor. Generally, “biogenerator” or “bioreactor” means a culture tank, generally made of stainless steel, or glass, with a volume of 0.5 liter or greater, comprising an agitation system, a device for injecting a stream of CO₂ gas and an oxygenation device. Typically, it is equipped with probes measuring the internal parameters of the biogenerator, such as the pH, the dissolved oxygen, the temperature, the tank pressure or certain physicochemical parameters of the culture (for instance the consumption of glucose or of glutamine or the production of lactate and ammonium ions). The pH, oxygen, and temperature probes are connected to a bioprocessor which permanently regulates these parameters. In another embodiment, the vessel is a WAVE Bioreactor (WAVE Biotech, Bridgewater, N.J., U.S.A.). Cell density in the culture may be determined by any method known in the art. For example, cell density may be determined microscopically (e.g., hemacytometer) or by an electronic cell counting device (e.g., COULTER COUNTER; AccuSizer 780/SPOS Single Particle Optical Sizer).

The term “infecting” means exposing the recombinant adenovirus to the cells or cell line under conditions so as to facilitate the infection of the cell with the recombinant adenovirus. In cells which have been infected by multiple copies of a given virus, the activities necessary for viral replication and virion packaging are cooperative. Thus, it is preferred that conditions be adjusted such that there is a significant probability that the cells are multiply infected with the virus. An example of a condition which enhances the production of virus in the cell is an increased virus concentration in the infection phase. However, it is possible that the total number of viral infections per cell can be overdone, resulting in toxic effects to the cell. Consequently, one should strive to maintain the infections in the virus concentration in the range of 10e6 to 10e10, preferably about 10e9, virions per ml. Chemical agents may also be employed to increase the infectivity of the cell line. For example, the present invention provides a method to increase the infectivity of cell lines for viral infectivity by the inclusion of a calpain inhibitor. Examples of calpain inhibitors useful in the practice of the present invention include calpain inhibitor 1 (also known as N-acetyl-leucyl-leucyl-norleucinal, commercially available from Boehringer Mannheim). Calpain inhibitor 1 has been observed to increase the infectivity of cell lines to recombinant adenovirus.

The term “culturing under conditions to permit replication of the viral genome” means maintaining the conditions for the infected cell so as to permit the virus to propagate in the cell. It is desirable to control conditions so as to maximize the number of viral particles produced by each cell. Consequently, it will be necessary to monitor and control reaction conditions such as, for example, temperature, dissolved oxygen and pH level. Commercially available bioreactors such as the CelliGen Plus Bioreactor (commercially available from New Brunswick Scientific, Inc. 44 Talmadge Road, Edison, N.J.) have provisions for monitoring and maintaining such parameters. Optimization of infection and culture conditions will vary somewhat, however, conditions for the efficient replication and production of virus may be achieved by those of skill in the art taking into considerations the known properties of the producer cell line, properties of the virus, and the type of bioreactor.

Virus, such as adenovirus, may be produced in the cells. Virus may be produced by culturing the cells; optionally adding fresh growth medium to the cells; inoculating the cells with the virus; incubating the inoculated cells (for any period of time); optionally adding fresh growth medium to the inoculated cells; and optionally harvesting the virus from the cells and the medium. Typically, when the concentration of viral particles, as determined by conventional methods, such as high performance liquid chromatography using a Resource Q column, as described in Shabram, et al. Human Gene Therapy 8:453-465 (1997), begins to plateau, the harvest is performed.

Fresh growth medium may be provided to the inoculated cells at any point. For example, the fresh medium may be added by perfusion. Medium exchange may significantly increase virus production in the cells. In one embodiment of the invention, the medium of cells is subject to two consecutive exchanges—one upon infection and another one day post-infection.

The cells used to produce the virus may be derived from a cell line frozen under serum-free medium conditions or from a cell line frozen under serum-containing medium conditions (e.g., from a frozen cell bank).

Suitable methods for identifying the presence of the virus in the culture, i.e., demonstrating the presence of viral proteins in the culture, include immunofluorescence tests, which may use a monoclonal antibody against one of the viral proteins or polyclonal antibodies (Von Bülow et al., in Diseases of Poultry, 10^{th} edition, Iowa State University Press), polymerase chain reaction (PCR) or nested PCR (Soiné t al., Avian Diseases 37:467-476 (1993)), ELISA (Von Bülow et al., in Diseases of Poultry, 10^{th} edition, Iowa State University Press)), hexon expression analyzed by flow cytometry (Musco et al. Cytometry 33:290-296 (1998), virus neutralization, or any of the common histochemical methods of identifying specific viral proteins.

Titrating the quantity of the adenovirus in the culture may be performed by techniques known in the art. In a particular embodiment, the concentration of viral particles is determined by the Resource Q assay as described by Shabram, et al. Human Gene Therapy 8:453-465 (1997). As used herein, the term “lysis” refers to the rupture of the virus-containing cells. Lysis may be achieved by a variety of means well known in the art. For example, mammalian cells may be lysed under low pressure (100-200 psi differential pressure) conditions, by homogenization, by microfluidization, or by conventional freeze-thaw methods. Exogenous free DNA/RNA may be removed by degradation with DNAse/RNAse.

The adenovirus-containing cells may be frozen. Adenovirus may be harvested from the virus-containing cells and the medium. In one embodiment, the adenovirus is harvested from both the virus-containing cells and the medium simultaneously. In a particular embodiment, the adenovirus producing cells and medium are subjected to cross-flow microfiltration, as described, for example, in U.S. Pat. No. 6,146,891, under conditions to both simultaneously lyse virus-containing cells and clarify the medium of cell debris which would otherwise interfere with virus purification.

The term “harvesting” means the collection of the cells containing the adenovirus from the media and may include collection of the adenovirus from the media. This may be achieved by conventional methods such as differential centrifugation or chromatographic means. At this stage, the harvested cells may be stored frozen or further processed by lysis and purification to isolate the virus. Exogenase free DNA/RNA may be removed by degradation with DNAse/RNAse, such as BENZONASE (American International Chemicals, Inc.).

The virus harvest may be further processed to concentrate the virus by methods such as ultrafiltration or tangential flow filtration as described in U.S. Pat. Nos. 6,146,891 and 6,544,769.

The term “recovering” means the isolation of a substantially pure population of recombinant virus particles from the lysed producer cells and optionally from the supernatant medium. Viral particles produced in the cell cultures of the present invention may be isolated and purified by any method which is commonly known in the art. Conventional purification techniques such as chromatographic or differential density gradient centrifugation methods may be employed. For example, the viral particles may be purified by cesium chloride gradient purification, column or batch chromatography, diethylaminoethyl (DEAE) chromatography (Haruna et al. Virology 13: 264-267 (1961); Klemperer et al., Virology 9: 536-545 (1959); Philipson et al., Virology 10: 459-465 (1960)), hydroxyapatite chromatography (U.S. Patent Application Publication Number US2002/0064860) and chromatography using other resins such as homogeneous cross-linked polysaccharides, which include soft gels (e.g., agarose), macroporous polymers “throughpores”, “tentacular” sorbents, which have tentacles that were designed for faster interactions with proteins (e.g., fractogel) and materials based on a soft gel in a rigid shell, which exploit the high capacity of soft gels and the rigidity of composite materials (e.g., Ceramic HyperD® F) (Boschetti, Chromatogr. 658:207 (1994); Rodriguez, J. Chromatogr. 699:47-61 (1997)). In the preferred practice of the invention, the virus is purified by column chromatography in substantial accordance with the process of Huyghe et al., Human Gene Therapy 6:1403-1416 (1995) as described in Shabram et al., U.S. Pat. No. 5,837,520 issued Nov. 17, 1998, and U.S. Pat. No. 6,261,823, the entire teachings of which is herein incorporated by reference.

Pegylation of Adenoviruses

PEG modification is a well-established technique for the modification of therapeutic peptides and proteins. A primary advantage of pegylation for proteins and peptides includes a reduction in antigenicity and immunogenicity. Preparation of PEG-protein conjugates requires, in general, activation of hydroxyl groups of PEG with a suitable reagent that can be fully substituted by nucleophilic groups (mainly lysine ε-amino groups) in the protein during the coupling reaction (O'Riordan et al., Hum. Gene Ther. 10:1349-1358 (1999)). A wide variety of methods has been developed to produce activated PEG linkers (Francis et al., J. Drug Target. 3:321-340 (1996)).

Likewise, many methods for conjugation of various polyethylene glycols to the capsid protein of adenoviruses are available ((O'Riordan et al., Hum. Gene Ther. 10:1349-1358 (1999)). Croyle et al. (Hum. Gene Ther. 11:1713-1722 (2000)) describes three conjugation methods with shortened reaction times that sufficiently modify the viral capsid and the physical stability of the adenovirus under extreme storage conditions. Other methods are available in the art.

A variety of activated polyethylene glycol linkers is available for the pegylation of proteins and rAds. PEG-SPA (SPA: succinimidyl ester of PEG propionic acid), which is polyethylene glycol activated with N-hydroxy succinimide (NHS) at one end and capped with a methoxy group at the other, may be selected to pegylate β-gal-rAd. This linker is available in consistently good quality, the reaction chemistry is relatively less complex than others such as Tresyl-PEG (TM-PEG), and the amide bond between the linker and lysine residue of rAd capsid proteins is stable for proteins such as IFNα-2b and IL-10. For example, 97% of pegylated IL-10 was stable to hydroxylamine when IL-10 was pegylated at pH 8.6 with PEG-SPA (data not shown).

PEG-SPA is also available with a fluorescein moiety on the PEG at the opposite end from the NHS ester. The advantage of this fluorescein-labeled PEG linker (here abbreviated as fluoro-PEG-SPA), is that it has the same reactivity as the PEG-SPA.

Other PEG linkers may also be used. For example, tresyl-MPEG (TM-PEG) has been used to successfully pegylate adenoviruses ((O'Riordan et al., Hum. Gene Ther. 10:1349-1358 (1999); Sigma Chemical (St. Louis, Mo.); Shearwater Polymers (Huntsville, Ala.); PolyMASC Pharmaceuticals (London, UK)). Other commercially available linkers include succinimidyl succinate MPEG (SS-PEG) and cyanuric chloride MPEG (CC-PEG) (Sigma Chemical Co. (St. Louis, Mo.).

General

In accordance with the present invention there may be employed conventional molecular biology, microbiology, and recombinant DNA techniques within the skill of the art. Such techniques are explained in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

Polynucleotide sequence,” or “nucleic acid sequence,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′O-methyl-, 2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR.sub.2 (“amidate”), P(O)R, P(O)OR, CO or CH.sub.2 (“formacetal”), in which each R or R′ is independently H or substituted or unsubstituted alkyl (1-20 C) optionally containing an ether (——O——) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, the term “coding sequence” or a sequence “encoding” refers to an expression product, such as a RNA, polypeptide, protein, or enzyme, is a nucleotide sequence that, when expressed, results in production of the product.

As used herein, the term “gene” means a DNA sequence that codes for or corresponds to a particular sequence of ribonucleotides or amino acids which comprise all or part of one or more RNA molecules, proteins or enzymes, and may or may not include regulatory DNA sequences, such as promoter sequences, which determine, for example, the conditions under which the gene is expressed. Genes may be transcribed from DNA to RNA which may or may not be translated into an amino acid sequence.

The term “stably integrated” means, with respect to an exogenous nucleic acid sequence, that such sequence is integrated into the genome of the cell such that successive generations of the cell retains the exogenous nucleic acid sequence.

The term “expression cassette” is used herein to define a nucleotide sequence capable of directing the transcription and translation of a heterologous coding sequence and the heterologous coding sequence to be expressed. An expression cassette comprises a regulatory element operably linked to a heterologous coding sequence so as to achieve expression of the protein product encoded by said heterologous coding sequence in the cell.

The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a coding sequence if it affects the transcription of the coding sequence. Operably linked means that the nucleotide sequences being linked are typically contiguous. However, as enhancers generally function when separated from the promoter by several kilobases and intronic sequences may be of variable lengths, some polynucleotide elements may be operably linked but not directly flanked and may even function in trans from a different allele or chromosome.

The term “regulatory element” refers to promoters, enhancers, transcription terminators, polyadenylation sites, and the like. The term “promoter” is used in its conventional sense to refer to a nucleotide sequence at which the initiation and rate of transcription of a coding sequence is controlled. The promoter contains the site at which RNA polymerase binds and also contains sites for the binding of regulatory factors (such as repressors or transcription factors). Promoters may be naturally occurring or synthetic. When the vector to be employed is a viral vector, the promoters may be endogenous to the virus or derived from other sources. The regulatory elements may be arranged so as to allow, enhance or facilitate expression of the transgene only in a particular cell type. For example, the expression cassette may be designed so that the transgene is under control of a promoter which is constitutively active, or temporally controlled (temporal promoters), activated in response to external stimuli (inducible), active in particular cell type or cell state (selective) constitutive promoters, temporal viral promoters or regulatable promoters.

A deficiency in a gene or gene function is a type of mutation which serves to impair or obliterate the function of the gene whose DNA sequences was mutated in whole or in part.

### EXAMPLES

The following examples are merely illustrative and not meant to be limiting of the scope of the invention described herein.

### Example 1

### Recombinant Adenovirus Vectors

The rAd vectors used in these pegylation studies were replication-deficient type 5 rAds either E1/E3-deleted and containing the human p53 transgene (p53 rAd) or E1-deleted containing the lacZ reporter gene ((β-gal rAd) (Wills et al., Hum. Gene Ther. 5:1079-1088 (1994)). The p53 rAd vector was produced in HEK 293 cells grown in bioreactors on Cytodex 3 microcarriers, the infected cells were separated from the beads by a fluidized bed, microfiltered to separate the cell debris, benzonase-treated, concentrated and diafiltered, then purified by anion exchange on DEAE-Fractogel and by gel filtration on Superdex 200 as previously described (Vellekamp et al., Hum. Gene Ther. 12:1923-1936 (2001)). The vector was in 14 mM Tris, 11 mM sodium phosphate, 2 mM MgCl₂, 2% sucrose, 10% (w/v) glycerol, pH 8.1 at 4° C. (Buffer A), unless otherwise noted. The (β-gal rAd (kindly provided by Dr. Robert Schnieder and produced at Canji, Inc.) was prepared from plaque purified isolates in D22-293 cells (Ling et al., Gene Ther. 9:907-914 (2002)) and then purified using column chromatography (Huyghe et al., Hum. Gene Ther 6:1403-1416 (1995)).

### Example 2

### Pegylation of rAd Using a Fluorescein-Labeled PEG-SPA and (Unlabeled) PEG-SPA

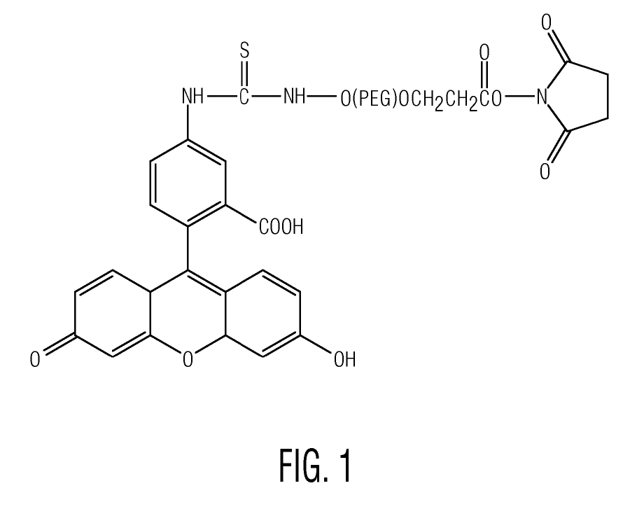

A variety of activated polyethylene glycol linkers is available for the pegylation of proteins and rAds. All work shown here used a linker with a PEG molecular weight of 5 KDa (FIG. 1). PEG-SPA (SPA: succinimidyl ester of PEG propionic acid), which is polyethylene glycol activated with N-hydroxy succinimide (NHS) at one end and capped with a methoxy group at the other, was used to pegylate β-gal-rAd. The details of the individual pegylation reactions are given in Table 1:

TABLE 1A
Pegylation reaction conditions for p53-rAd with fluoro-PEG-SPA

| p53-rAd | | fluoro-PEG- | | |
|---|---|---|---|---|
| concentration | Tris concen- | SPA concen- | reaction pH | |
| particle/ml | tration, mM | tration (w/v) % | initial pH | final pH |
| 0.55 × 10¹² | 149 | 1.3 | 9.0 | 8.91 |
| 0.55 × 10¹² | 149 | 2.5 | 9.0 | 8.82 |
| 0.55 × 10¹² | 149 | 5.0 | 9.0 | 8.65 |
| 0.55 × 10¹² | 149 | 7.4 | 9.0 | 8.34 |
| 0.55 × 10¹² | 149 | 10.0 | 9.0 | 8.16 |
| 0.91 × 10¹² | 140 | 2.7 | 9.0 | 8.62 |
| 0.91 × 10¹² | 140 | 5.1 | 9.0 | 8.45 |
| 0.91 × 10¹² | 140 | 9.9 | 9.0 | 8.15 |
| 0.85 × 10¹² | 222 | 1.04 | 8.33 | 8.32 |
| 0.85 × 10¹² | 222 | 2.83 | 8.33 | 8.24 |
| 0.85 × 10¹² | 222 | 5.91 | 8.33 | 8.16 |
| 0.85 × 10¹² | 222 | 7.43 | 8.33 | 8.04 |
| 0.85 × 10¹² | 222 | 10.4 | 8.33 | 7.90 |
| 0.5 × 10¹² | 52 | 2.0 | 8.12 | 7.92 |
| 0.5 × 10¹² | 91 | 2.0 | 8.18 | 7.99 |
| 0.5 × 10¹² | 163 | 2.0 | 8.24 | 8.03 |

TABLE 1B
Pegylation reaction conditions for β-gal-rAd with PEG-SPA

| β-gal-rAd | | | | |
|---|---|---|---|---|
| concentration, | Tris concen- | PEG-SPA concen- | reaction pH | |
| particle/ml | tration, mM | tration (w/v) % | initial pH | final pH |
| 0.58 × 10¹² | 56 | 1.0 | 8.9 | 8.7 |
| 0.55 × 10¹² | 136 | 4.0 | 9.0 | 8.82 |
| 0.55 × 10¹² | 136 | 8.0 | 9.0 | 8.69 |

The concentrations for rAd, Tris, and linkers were nominal ones, based upon the reaction volumes prior to the addition of linker powder into a reaction solution. Their actual concentrations are dependent on the volume increase caused by the linker powder dissolution, but the maximum dilution effect, which occurred with 10% (w/v) linker used, was about 8%. The reaction volumes were 2 ml and 60 ml, for the fluoro-PEG-SPA and PEG-SPA linkers, respectively. Fluoro-PEG-SPA, which is also called fluorescein-PEG-NHS (Nektar catalog #IK2ZOH02), and PEG-SPA (Nektar catalog #2M4MOH01) were purchased from Nektar Therapeutics, San Carlos, Calif., (formally Shearwater Polymers, Inc). In general, aliquots of rAd (frozen at −80° C.) were thawed and mixed at room temperature with a small volume of concentrated Tris-Cl (1.5 M) to increase the reaction pH and buffering capacity.

The basic rAd solutions were divided into equal aliquots and each aliquot was mildly agitated using a stir bar in a glass reactor. The pegylation reaction was initiated by adding a predetermined amount of linker as powder into each aliquot. The stirring continued at room temperature for one hour. The final reaction pH ranged from 7.9 to 8.9, depending on the reaction condition.

PEG-SPA (also called PEG-NHS) in a pegylation reaction undergoes two pathways, aminolysis and hydrolysis. In aminolysis, the linker reacts with the unprotonated amino group of a protein or potentially a reaction component such as the Tris used here. This results in pegylated protein or pegylated Tris, conjugated via an amide bond. NHS is released from the linker as a reaction by-product. In hydrolysis, the linker reacts with water to release NHS. Each pathway causes reaction pH to drop.

However, unlike PEG-SPA, the fluorescein moiety of the fluorescent linker (fluoro-PEG-SPA) has a carboxylic acid group (see FIG. 1). As a result, control of the pegylation reaction pH is more difficult with fluoro-PEG-SPA than with PEG-SPA, i.e., the fluorescent linker itself causes an additional drop in a reaction pH as it dissolves in the virus solution. In Table IA, the initial and final pHs for all the pegylation reactions with fluoro-PEG-SPA are shown. The reaction pH range of 7.9 to 9.0 employed in this work was also a stable pH range for rAd. The initial pH was that of the virus solution prior to the fluoro-PEG-SPA addition and the final pH was the reaction pH one hour after the solid linker was added. The linker fully dissolved in the virus solution within three minutes even at the highest linker concentration. Due to the immediate sharp pH drop caused by physical dissolution of the acidic linker, the average virus exposure to the range of the reaction pH should be less than the difference between the initial and final pH values. For example, there was no significant difference in reaction pH from 0.5 to 1.0 hour. No glycine was added to quench the pegylation reaction since virtually no active linker remains after one hour due to the aminolysis and hydrolysis reactions. The linker half-life at pH 8.0 at 25° C. is 16.5 minutes due to hydrolysis alone. Resource Q analysis of reaction time-point samples showed that a pegylation reaction was complete within one hour and there was no loss of virus under these conditions (data not shown).

Following the pegylation reactions, the aliquots were frozen at −80° C. for subsequent purification.

### Example 3

### Purification of the Fluorescein-Labeled and Unlabeled Pegylated rAds

The frozen virus reaction solutions were thawed and the pegylated virus vectors were separated from the reaction by-products using gel filtration chromatography. For the fluoro-PEG-rAds (using p53-rAd), 0.5 ml to 1.0 ml of each reaction solution was loaded onto a Superdex 200 HR 10/30 column equilibrated and run in Buffer A at room temperature. For PEG-rAd (using β-gal-rAd), the reaction solution (23-28 ml) was loaded onto Superdex 200 Prep grade 26/60 column run at 4° C. also with Buffer A. The chromatography of each pegylated rAd was monitored at 260 nm, and the PEG-rAd peak, which eluted at the void volume, was collected. Each pool was immediately filtered using 0.2 um syringe filters, and aliquots were frozen at −80° C.

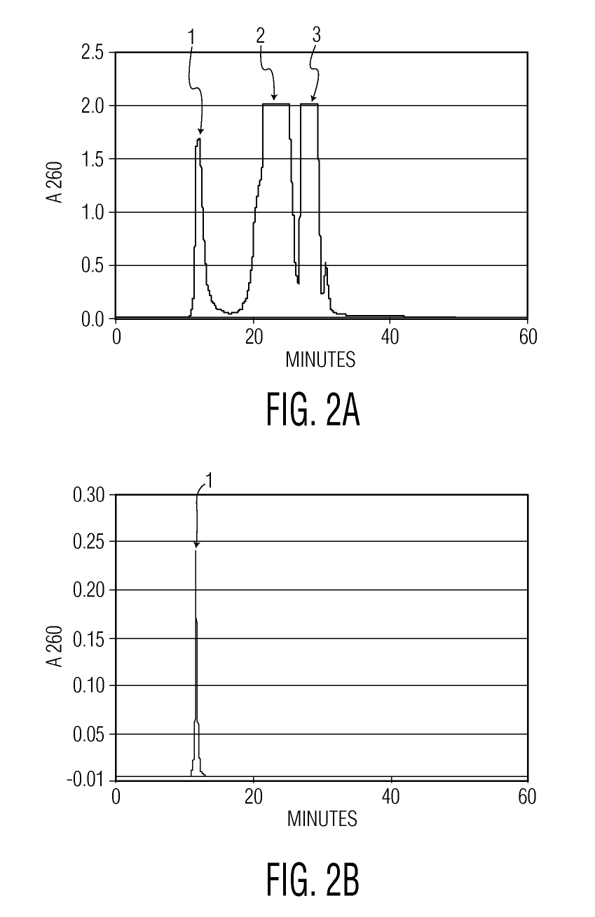

FIG. 2A shows a typical purification chromatography absorbance profile monitored at 260 nm. The first peak is the pegylated rAd eluting at the column void volume. The second peak contains potentially three PEG related molecules: fluoro-PEG from hydrolysis, fluoro-PEG-Tris, and any trace unreacted fluoro-PEG-SPA. The third peak is NHS. The virus peak was narrowly and symmetrically pooled. The purification yield was 60% to 70% based on A260 in 0.1% SDS and confirmed with Resource Q HPLC.

### Example 4

### Fluorescence Size Exclusion HPLC (SE-HPLC) Assay

The purified fluoro-PEG-rAds were injected on SE-HPLC to check purity based on A260. A representative chromatogram is shown in FIG. 2B. Typical purity was more than 98%. Purified fluoro-PEG-rAd was frozen at −80° C. for subsequent DP estimation on fluorescence SE-HPLC.

A Superdex 200 H/R 10/30 column was used at room temperature to separate and measure the fluorescence intensity of fluoro-PEG-rAd. The running buffer was 20 mM NaPi, 10 mM TrisHCl, 100 mM NaCl, 2 mM MgCl₂, 0.1% (v/v) Triton X-100 at pH 8.1. The HPLC system (Waters Corp., Milford, Mass.) included a Waters 474 scanning fluorescence detector with excitation and emission wavelengths set at 490 nm and 520 nm, respectively. The fluoro-PEG-SPA linker that was used above for the pegylation reaction (substitution and purity>96%) was dissolved in the running buffer and used as a standard to measure the number of molecules of fluoro-PEG attached to virus particles in a fluoro-PEG-rAd peak. Purified fluoro-PEG-rAd was diluted 20-fold with the running buffer and 10-40 μl of the resultant samples were injected to SE-HPLC.

### Example 5

### Determination of the Degree of Pegylation of the Flourescein-Labeled Pegylated rAd by Size Exclusion HPLC Fluorescent Detection Assay

The degree of pegylation (DP) was calculated as the ratio of the number of fluoro-PEG molecules displayed by a fluoro-PEG-rAd peak to the number of fluoro-PEG-rAd particles injected to SE-HPLC. The column was routinely regenerated with 0.5 N NaOH after each 20 injections or whenever there was deterioration in column performance determined by reduced fluorescence signal of fluoro-PEG-rAd.

The fluoro-PEG-rAd vectors purified using gel-filtration chromatography were sufficiently pure that a spectrofluorometer could quantify the amount of the fluorescein-labeled PEG bound to the virus. In this study, however, fluorescence size exclusion HPLC (SE-HPLC), that is, SE-HPLC run on a system with a fluorescent in-line detector was employed. This ensured that the quantified fluorescence signal was emitted by the fluoro-PEG-rAd peak, not by any other potential fluorescence impurity peaks such as free fluorescein or fluorescein-PEG-related molecules.

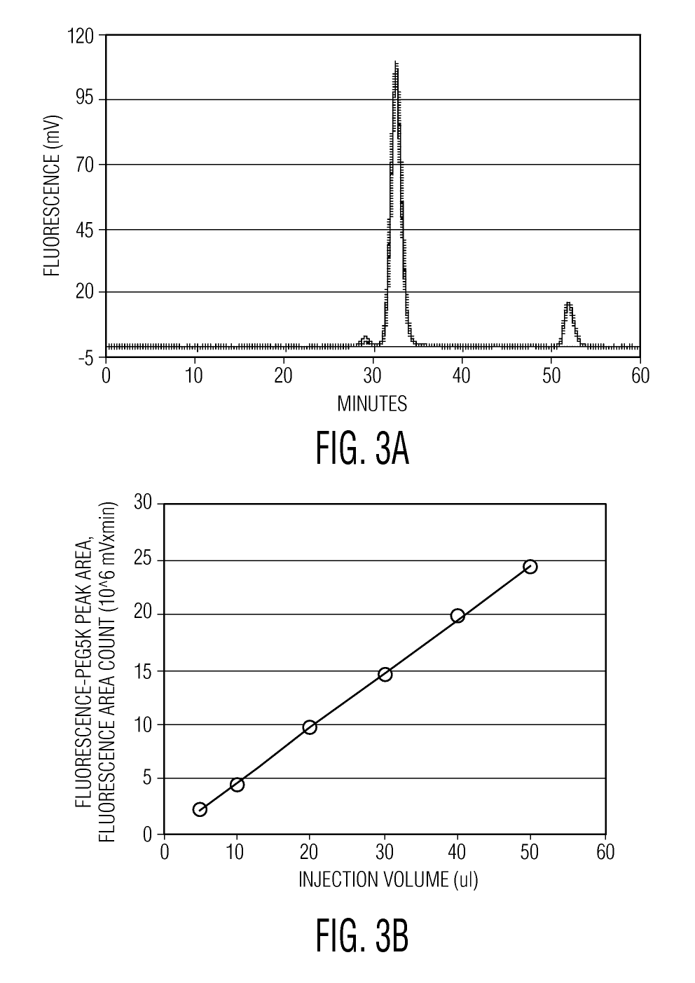

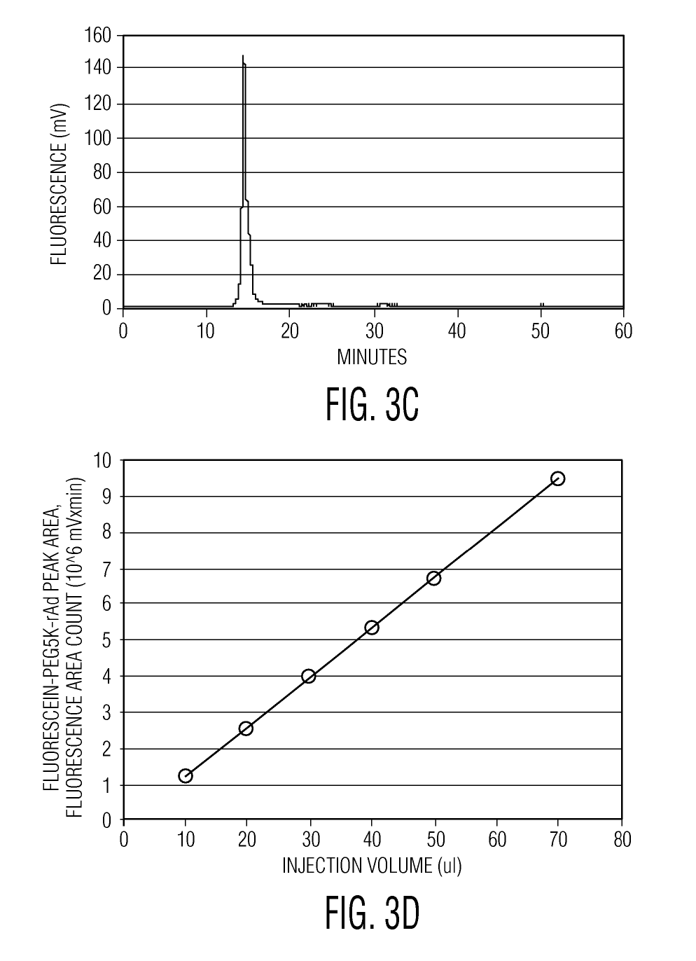

To establish the value of the fluorescence SE-HPLC assay, the fluoro-PEG was first analyzed as a standard for linearity and reproducibility. To prepare the standard, the fluoro-PEG-SPA was dissolved in the fluorescence SE-HPLC running buffer (pH 8.1). During the dissolution at room temperature, essentially all of the fluoro-PEG-SPA was hydrolyzed to fluoro-PEG. A typical fluorescence elution profile of this standard is shown in FIG. 3A. The peak area of the fluorescence signal of fluoro-PEG (and any trace fluoro-PEG-SPA) was highly linear with respect to the injection volume over a wide range (FIG. 3B). The fluoro-PEG standard was also injected without the SE column in-line, bypassing the column, to detect any potential losses to the column. The fluorescent signal was only about 3% higher, which indicated that, given baseline considerations, there was no loss of the standard to the column (data not shown). Day-today reproducibility of the standard curve was very high. A sample of the fluoro-PEG standard was kept at pH 8.0 and 4° C. for approximately 10 days prior to analysis. Its chromatogram was compared to that of the freshly-prepared standard (FIG. 3A). There was no significant change of fluoro-PEG peak area though a minor impurity peak of higher apparent MW disappeared and a small late-eluting peak that likely is free fluorescein appeared. This showed that this standard was stable at 4° C. and could be used to quantify the fluorescence signal of fluoro-PEG-rAd. The purified fluoro-PEG-rAd samples were diluted 20-fold with the running buffer and then run on the fluorescent SE HPLC. A typical profile (FIG. 3C) shows essentially all the signal eluted in the virus peak position. Linearity of the rAd fluorescence signal with respect to injection volume was also observed (FIG. 3D). These results, compared with injections of the fluoro-PEG-rAd which bypassed the column, showed that there was less than 10% loss of fluorescein-labeled rAds to the column when the column was relatively new and sufficiently cleaned and equilibrated (data not shown). Loss of fluorescent signal to the column of greater than a 10% should be addressed.

Except for one fluoro-PEG-rAd preparation, the purity as determined by fluorescence SE-HPLC was routinely greater than 97%. This one sample, prepared at the highest linker concentration (10.4%), was 88% pure with a 12% fluorescein-PEG impurity. This reflects that with the higher linker concentrations in the pegylation reaction, the baseline resolution of the linker from the pegylated rAd on purification by gel filtration is more challenging. We also noted that when the fluorescent SE HPLC profile scale of these samples was greatly expanded a minor trailing shoulder of the pegylated rAd peak was observed (see stability section below). This slight asymmetry was relatively unaffected by the degree of pegylation.

For each fluoro-PEG-rAd preparation, the average number of PEG molecules conjugated per rAd particle, or the degree of pegylation (DP), was determined. Using the fluorescence SE HPLC profile, the peak area of rAd fluorescence was converted to the equivalent amount of fluoro-PEG using the standard curve and then divided by the amount of rAd particles injected based on the A260 of the sample in 0.1% SDS (after the contribution of fluorescein moiety to A260 was subtracted as described above).

### Example 6

### Control Pegylation of rAd Using Fluoro-PEG (Inactive Linker)

To confirm that the fluorescence-label of the PEG was bound to the rAd only by pegylation through the SPA moiety, a negative control pegylation reaction was performed with inactivated linker. The inactivation of fluoro-PEG-SPA due to aminolysis and hydrolysis was achieved by addition of the linker to a pegylation reaction mixture lacking rAd followed by incubation for 5 hours at 25° C. It is estimated that this would decrease the initial nominal active linker concentration from 30 mg/ml to <0.1 ug/ml. (Under these conditions, pH 8.0 and 25° C., the half-life of the linker due to hydrolysis is about 17 minutes (Nektar Therapeutics catalogue) and virtually all, i.e., 99.9995%, of the active linker would be hydrolyzed.) Considering the additional loss of the active linker due to aminolysis the remaining active linker was estimated as less than 0.0005%. Therefore, the active linker concentration was less than 0.1 ug/ml although the initial nominal linker concentration was 30 mg/ml.) The rAd was then added into the inactivated-linker solution to start a pegylation reaction as a negative control experiment. After one additional hour the rAd was purified from the reaction mixture by gel filtration chromatography.

Analysis of this sample by fluorescent SE HPLC gave a value of only eight fluoro-PEG per rAd particle (Table 2). This, however, is only negligibly more than the intrinsic fluorescent signal of the rAd itself which was equivalent to six fluoro-PEG per rAd particle. This indicates that there is essentially no association of the fluorescent reaction by-products, fluorescein-PEG, fluorescein-PEG-Tris, or fluorescein, with rAd viral particles, and that these fluorescent pegylation reaction by-products do not interfere with fluorescent SE HPLC assay.

TABLE 2
Pegylation of rAd using inactivated fluorescence linker

| | particles | # of Fluoro-PEG | Fluoro-molecules |
|---|---|---|---|
| Sample | injected | in virus peak | per particle |
| unmodified rAd | 2.50E+09 | 1.54E+10 | 6 |
| modified rAd | 0.99E+09 | 0.81E+10 | 8 |
| with inactivated | | | |
| linker at 2% | | | |

### Example 7

### Determination of Virus Particle Concentration of Fluorescein-PEG-rAd

For unmodified rAd, the virus particle concentration was determined by A260 in 0.1% SDS; 1.0 A260 was equivalent to 1.1×10¹² particles/ml (Maizel, Jr. et al., 1968). In the case of fluorescein-PEG-rAd, the determination of the virus concentration was discounted for the A260 contribution of fluorescein-PEG molecules attached to rAd. The extinction coefficient of fluorescein-PEG attached to rAd was assumed to be equal to that of fluorescein, 2.5×10⁻¹⁷(A260×ml/molecule). The total amount of fluorescein-PEG per ml in a sample was measured on fluorescent SE-HPLC and its contribution to the observed A260 was its extinction coefficient (A260×ml/molecule)×fluorescein-PEG concentration (molecules/ml). Therefore, the net A260 of the virus used to estimate the particle concentration was the total A260 of fluorescein-pegylated rAd minus the A260 of fluorescein-PEG. Obviously this concentration correction was more significant with more highly fluorescent-pegylated rAd. At 2,500 DP, the correction was about 8%.

### Example 8

### Characterization of Pegylated rAd with Various Degrees of Pegylation by SDS-PAGE

Having established the DP of the fluoro-PEG rAds (Example 5), a set of these purified samples prepared at differing linker concentrations were compared to a set of previously prepared, non-fluorescent PEG-rAds (Table 1B) using a variety of non-fluorescent analytical techniques. Both similarities and differences were observed.

Pegylated rAds were analyzed by SDS-PAGE assay as described previously (Vellekamp et al., Hum. Gene Ther. 12:1923-1936 (2001)). Pegylated or unmodified (control) rAd were loaded onto Novex SDS precast 4-20% acrylamide gradient gels, then run and stained according to manufacturer recommendations. Gel image scanning on Coomassie blue-stained gels for quantification of the relative amounts of the hexon bands in different pegylated rAd preparations (˜0.3-0.5×10¹⁰ particles per lane) used a Molecular Dynamics scanner with the automatic baseline method. Peak heights of the individual stained bands were normalized for small differences in the amount of particles loaded. The hexon band intensity (peak height) is not strictly linear at higher hexon concentrations. The fluorescent image was captured immediately after completion of the electrophoresis on the Kodak Image Station 440CF using UV/fluorescent detection mode with filter #61, a band pass filter of 498-568 nm, with an f-stop setting of 2; it was exposed twice at 15 seconds each for a total of a 30 second exposure.

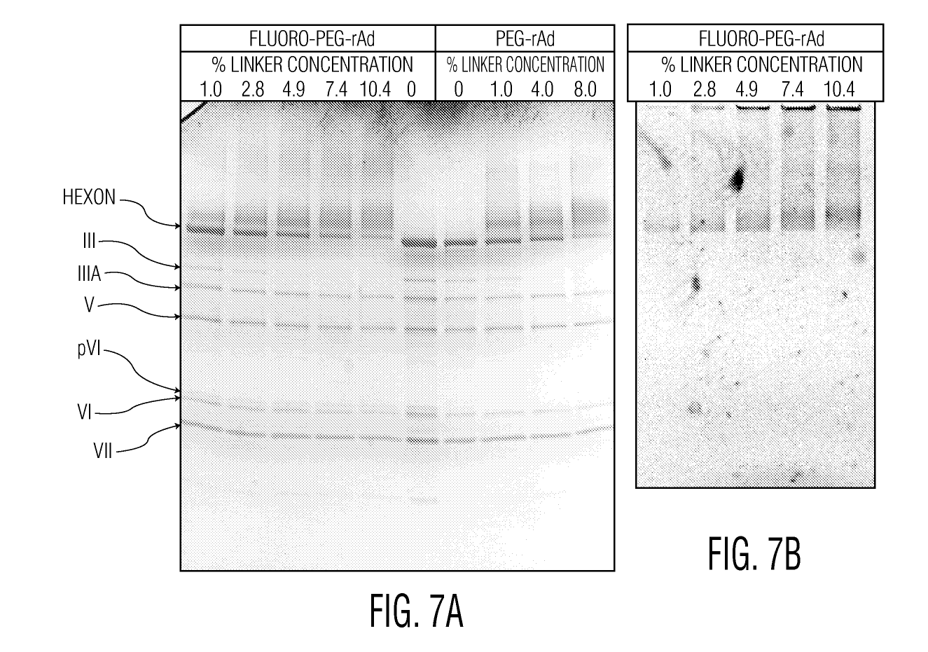

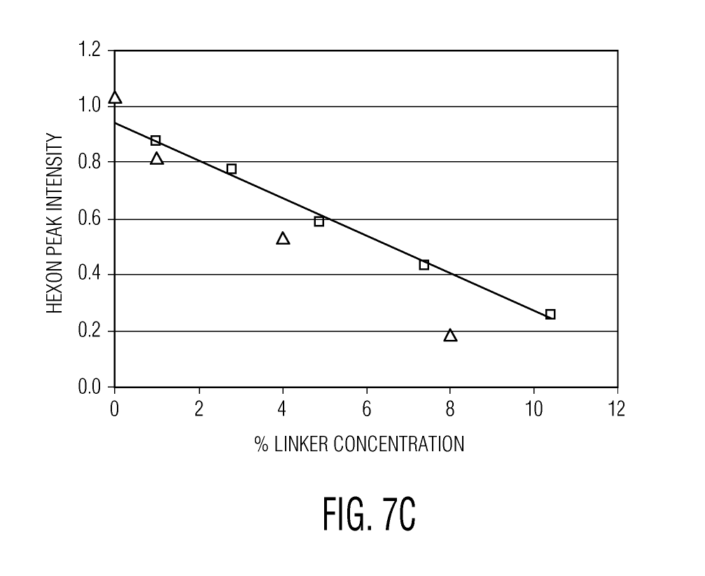

Increased pegylation of rAds showed decreasing amounts of hexon monomer and III (penton base) on SDS-PAGE (FIG. 7A). No change was observed in the amounts of proteins IIIa, V, VI, and VII, or in pVI, the precursor to VI, pVI is a major component of the empty capsids (Vellekamp et al., Hum. Gene Ther. 12:1923-1936 (2001)), which are present at approximately 10% particle concentration in the initial p53-rAd used to prepare the fluoro-PEG-rAd. The loss of the hexon monomer band seen with increasing pegylation of the fluoro-PEG rAd was accompanied by an increasing diffuse protein band that migrates at an approximately 20-50K higher molecular weight position that likely represents the mono-pegylated or multi-pegylated hexon monomers. The pegylated rAds also showed a faint diffuse band at the approximate position of the hexon trimer. This suggests that pegylation may somewhat stabilize the trimer form. The Coomassie stained gel was also imaged for fluorescence (FIG. 7B). This image again detected the increasing amounts of pegylated hexon monomer and the faint diffuse band of hexon trimer with increased pegylation of rAd. Some increasing fluorescence was detected at the top of the gel which also suggests that the pegylated virus were more resistant to complete disruption by heating in the SDS-PAGE sample buffer.

The amount of hexon in each lane of FIG. 7A was determined by scanning densitometry. The value of the hexon peak height, normalized by the amount of rAd particles loaded, was plotted versus the percent linker concentration used in the preparation of that sample (FIG. 7C). Both the fluoro-PEG-rAd and the PEG-rAd show a strong dependence of the hexon loss on the % linker concentrationin the pegylation reaction. However this relationship differs between the two pegylated vector types. For example, the PEG-rAd prepared at 4% linker concentration demonstrates a loss of hexon equivalent to a fluoroPEG-rAd prepared at 6% linker. This indicates that the PEG-rAd vectors used here have a detectably higher DP than the fluoro-PEG-rAd vectors when produced at the same % linker concentration.

### Example 9

### Characterization of Pegylated rAd with Various Degrees of Pegylation by Anion Exchange Chromatography

A set of the purified fluoro-PEG-rAd samples (Example 5) prepared at differing linker concentrations were next compared to a set of previously prepared, non-fluorescent PEG-rAds (Table 1B) using anion exchange chromatography.

The anion exchange assay by Resource Q chromatography (Shabram et al., Hum. Gene Ther. 8:453-465 (1997)) was used to determine the virus particle concentration as well as to characterize the pegylated rAds.

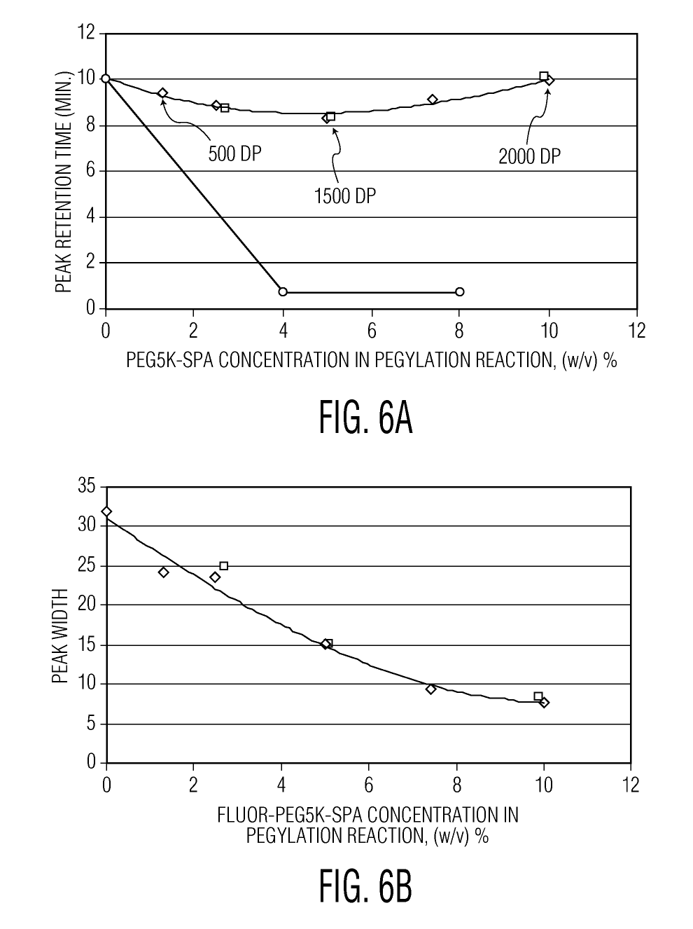

The most striking difference between the fluoro-PEG-rAds versus the PEG-rAds was observed with the anion exchange assay on Resource Q (FIG. 6A). For the PEG-rAds, as the degree of pegylation increased, the peak retention time rapidly decreased such that PEG-rAd prepared at 4% linker concentration completely failed to bind to the column. This was interpreted as the shell of PEG that surrounds the virus increased, it shielded the negative charge on the rAd from interacting with the positive charge of the anion exchange column. The fluoro-PEG rAds at the lower linker concentrations showed a similar but much weaker trend such that fluoro-PEG-rAd prepared at 4% linker still completely bound. However with the fluoro-PEG samples prepared at still higher linker concentrations the peak retention times increased. This very different elution behavior was interpreted as resulting from the competing effects of the PEG shielding of the virus particle's negative surface charge versus the addition of negative charge at the exterior of the PEG shell from the anionic carboxylate moiety of the fluorescein at the PEG terminal. Also noted was the broadening of the peaks of samples prepared at increased linker concentrations (FIG. 6B), suggesting a heterogeneity in the binding interactions of the fluoro-PEG rAds. Independent of these changes in peak retention time and width, the area of all the peaks was proportional to the rAd amount injected on the column and was therefore useful as a rAd quantification tool.

### Example 10

### Characterization of Pegylated rAd with Various Degrees of Pegylation by RP-HPLC

A set of the purified fluoro-PEG rAd samples (Example 5) prepared at differing linker concentrations were next compared to a set of previously prepared, non-fluorescent PEG-rAds (Table 1B) using RP-HPLC. The RP-HPLC analysis was performed using a modification (Sutjipto et al., Hum Gene Ther. 16(1):109-25 (2005)) of the method originally described (Lehmberg et al, J. Chromatogr. B. Biomed. Sci. Appl. 732(2)411-23 (1999)).

Like SDS-PAGE, analysis with RP-HPLC of both fluoro-PEG-rAd and PEG-rAd showed a decline in the hexon and III (penton base) peaks with pegylation at increasing % linker concentrations (data not shown). The loss of the hexon peak coincided with the increase of one to three additional peaks closely following the hexon peak. These peaks are likely mono-, di-, and tri-pegylated forms of the hexon monomer. These poorly resolved peaks are difficult to individually quantify but comparative estimations of the relative hexon DP would be consistent with the results of the fluorescent and non-fluorescent pegylated rAds from SDS-PAGE and AUC. Future modification of the RP-HPLC method could make this a useful technique for the determination of the DP of pegylated rAds. No other rAd protein peaks were observed to disappear with increased % linker concentration (fiber is not routinely detected with RP-HPLC) with the exception of a limited decline in the amount of protein IX, a capsid protein, in the PEG-rAd.

### Example 11

### Characterization of Pegylated rAd by Analytical Ultracentrifugation on CsCl Gradients

Samples of PEG-rAd with varying DP and the β-gal rAd control were examined separately on analytical ultracentrifugation in CsCl density gradients.

Sedimentation equilibrium experiments of the rAd forms in CsCl gradients were performed using a Beckman Optima XL-A analytical ultracentrifuge equipped with the scanning UV-absorption and computer data capture (Yang et al., 2003). Pegylated rAd and rAd in Buffer A were diluted to approximately 0.1-0.6×10¹² particle/ml and mixed with 456 mg CsCl per ml of rAd sample. This concentration was selected to give a solution density of approximately 1.32 g/ml (including contributions to the density from the glycerol and sucrose present in the samples) which is intermediate between the densities of the empty capsids and the complete virus. The samples (420 μl) were loaded to the sample channels in 2-channel epon centerpieces of 12 mm optical path length assembled in the cell housing unit. The reference channels were filled with the Buffer A plus CsCl solution. Centrifugation was run in an An-50 Ti eight-place rotor at 4° C.

Following installation of cell units in the rotor, the rotor was first brought to 3000 RPM to provide a background scan of the sample absorbance, then was brought to 40,000 rpm except where indicated. All samples were scanned for absorbance at 260, 280 and 320 nm in a continuous mode at time intervals as desired. Typically, samples were run for 16 hours and scans were taken at 8 and 16 hours after the initial high speed scan. UV profiles were plotted using Origin 6.0.

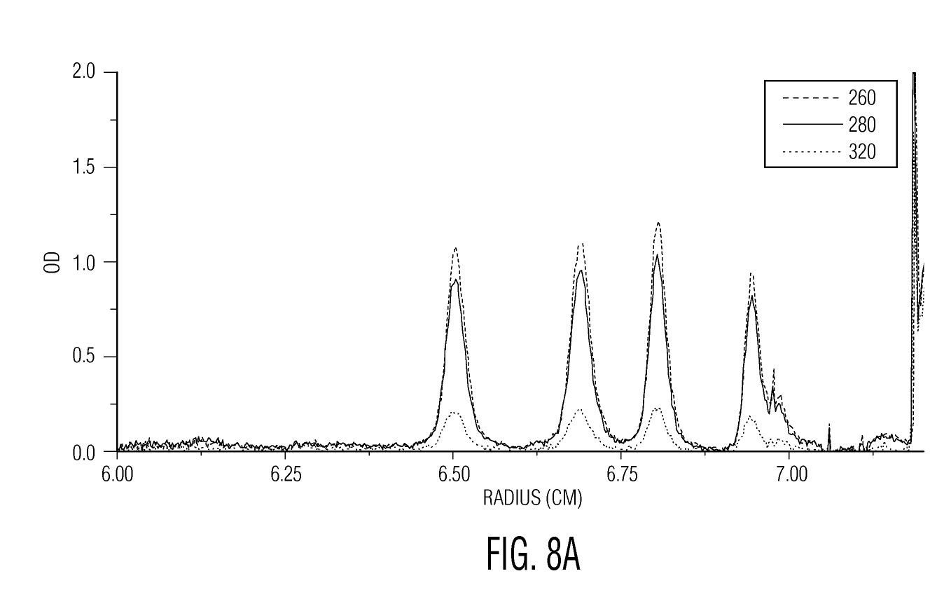

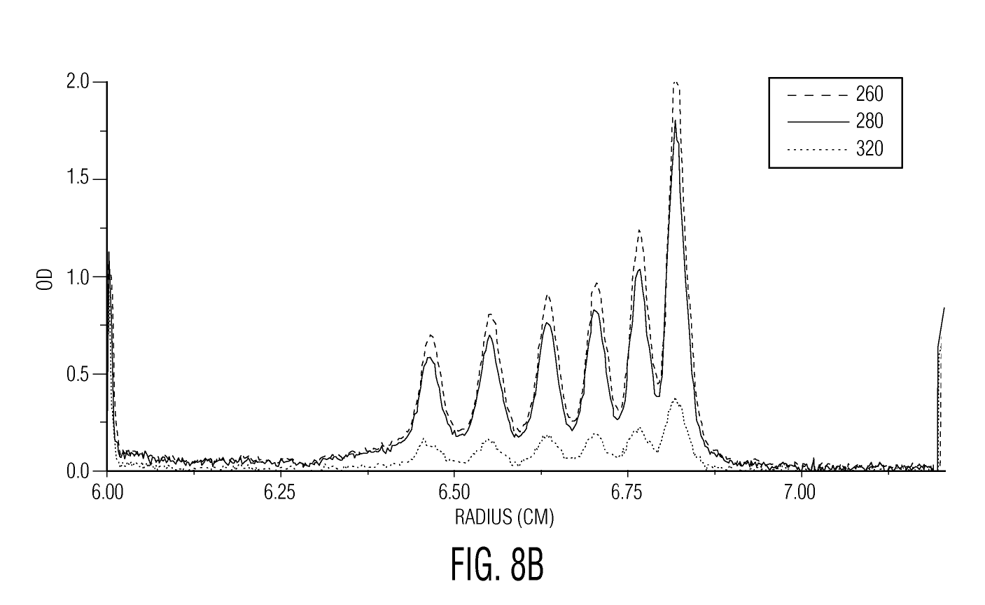

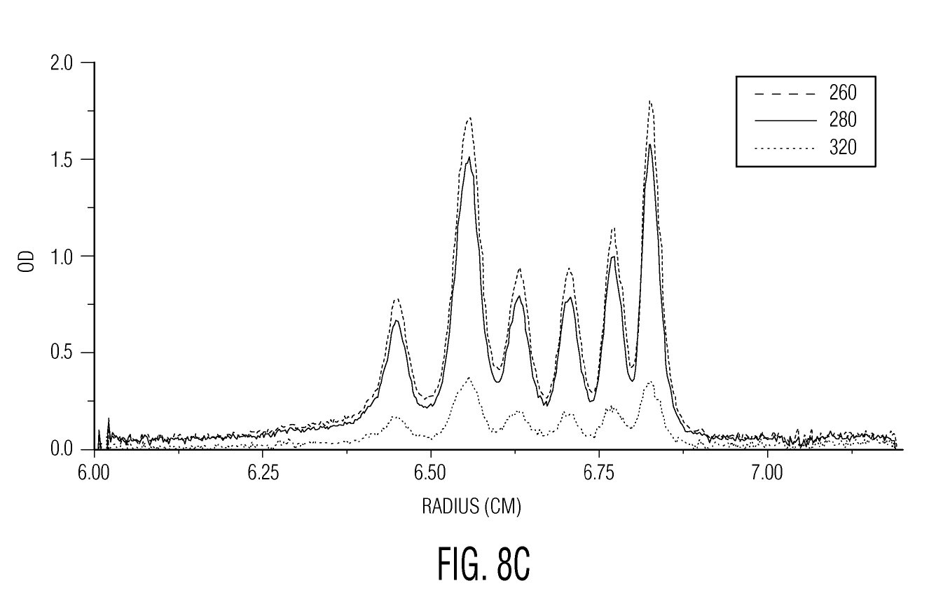

Each rAd preparation displayed a single UV peak of similar shape but the density position of each peak was altered with the rAd of the highest DP showing the lowest density and the β-gal rAd control (no pegylation) showing the highest density (data not shown). To confirm these density differences, a mixture of the β-gal rAd control and PEG-rAds (1%, 4%, and 8% linker concentration in the pegylation reaction) was prepared at a concentration of 3×10¹⁰ particles/ml of each. The resulting AUC profile after 16 hours at 30,000 RPM shows four well resolved peaks (FIG. 8A). The lower centrifugation speed optimized the separation by reducing the CsCl density gradient slope. A profile after 40 hours centrifugation indicated that these pegylated vectors were stable in high concentrations of CsCl, even in the approximately 20-fold virus-concentrating environment of the AUC.

The set of fluoro-PEG-rAd vectors (1.0%, 2.8%, 4.9%, 7.4%, and 10.4% linker concentration in the pegylation reaction) were also mixed together at a concentration of 3×10¹⁰ particles/ml of each (including the unmodified control at 4.5×10¹⁰ particles/ml) was run on AUC as above. This profile showed six well resolved peaks (FIG. 8B). Since the peak positions of AUC with CsCl gradients can be sensitive to very small differences in the sample volume and CsCl addition it was necessary to compare the peak positions of the fluorescent and non-fluorescent pegylated vectors in FIGS. 8A and 8B. This was done by taking the mixture of fluoro-PEG rAds in FIG. 8B at a reduced rAd concentration and adding to it just the non-fluorescent PEG-rAd prepared at the 4% linker concentration as in FIG. 8A. This mixture was analyzed as above with the resulting profile shown in FIG. 8C. The non-fluorescent PEG-rAd prepared at the 4% linker concentration clearly displayed a buoyant density very similar to that of the fluoro-PEG-rAd prepared at the 7.4% linker concentration. Additional AUC experiments that varied the initial PEG-rAd concentration from 1 to 4×10¹¹ particles/ml together with a very low concentration of the β-gal rAd control (5×10⁹ particles/ml) showed that the density position was independent of the PEG-rAd concentration (data not shown).

The striking difference of the rAd particle density with the different degree of pegylation is likely due to the averaging of the low density shell of polyethylene glycol together with the high density virus particle within. Thus AUC reveals itself as a further technique for the evaluation of the relative degree of rAd pegylation and suggests that preparative CsCl density gradient centrifugation would be an alternative PEG-rAd purification technique. Furthermore this AUC data in FIG. 8 demonstrates that the conditions of the pegylation reaction itself lead to a relatively narrow distribution in the degree of pegylation of the rAd. Estimating from the AUC profile peak width at 320 nm run, >90% of all particles in any sample are much less than +/−100 DP of the estimated DP of the particular sample.

### Example 12

### FACS-Based Infectivity Assays

The measurement of infectious particle concentration by flow cytometry has been reported previously (Zhu et al., Cytometry 1 37(1):51-59 (1999)). Briefly, human 293 cells are infected with a limited amount of virus and at 48 hours post-infection the cells are fixed, treated with a fluorescent-labeled anti-hexon monoclonal antibody, and the fluorescent cells per 50,000 total cells are counted using a FACSCalibur flow cytometer (Becton Dickinson).

Samples with the full range of DPs of both the fluoro-PEG-rAd and PEG-rAd were analyzed with a FACS-based infectivity assay which evaluates the infected 293 cells for hexon production using a fluorescent-labeled anti-hexon monoclonal antibody. Both types of pegylated vectors were essentially noninfectious, losing 2-3 logs of infectivity, using this assay. This is not unexpected since the uptake by these cells is based on the coxsackie adenovirus receptor-mediated uptake through binding to the virus fiber protein and this protein is likely blocked by the PEG moiety. However the PEG-rAd β-gal rAd) vectors were active in transducing tissue with in vivo mouse models (Dr. Drake LaFace; unpublished data).

### Example 13

### Effects of Pegylation Reaction Conditions on the Degree of Pegylation of rAd

The method described in Example 4 was next used to evaluate the effect of reaction variables on the degree of pegylation.

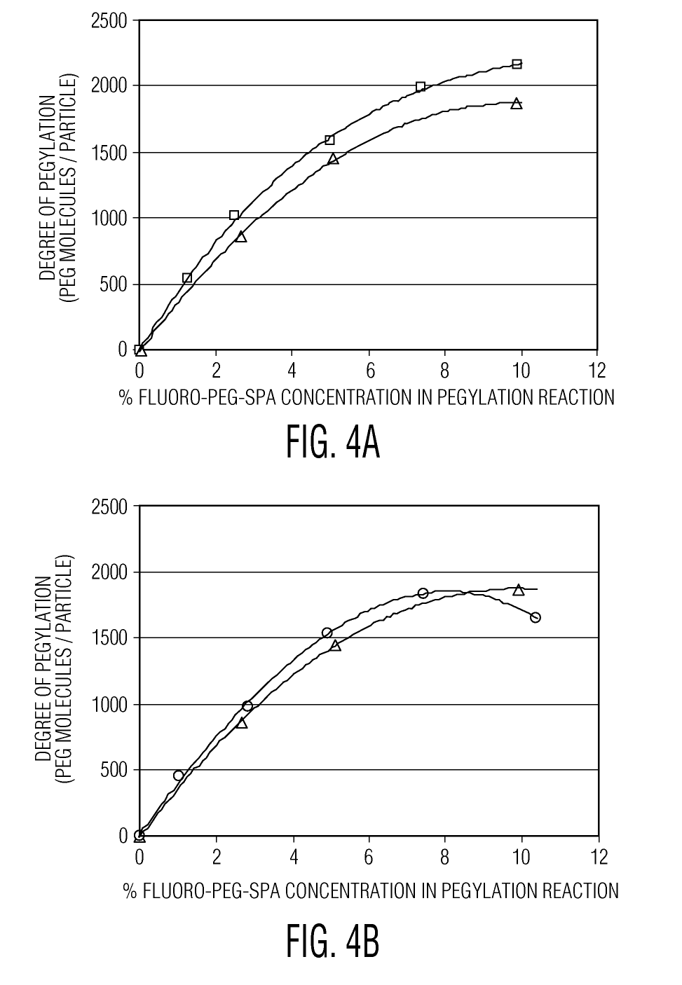

Linker concentration Samples of the fluoro-PEG-rAd prepared according to Example 2 under the reaction conditions listed in Table 1A were used to determine their DP. This was graphed for each of three sets versus the percent linker concentration used in its reaction (FIG. 4). For example, the reaction samples prepared at an rAd concentration of 0.55×10¹² particles/ml were determined to have DPs of 540, 1000, 1590, 1990, and 2170 at 1.3%, 2.5%, 5.0%, 7.4% and 10.0% linker concentration, respectively. All sets showed an almost linear dependence on linker concentration up to about 1500 DP with declining dependence between 1500-2500 DP suggesting a saturation of the potential pegylation sites on the virus by the fluoro-PEG at these higher linker concentrations.

Virus concentration Since the pegylation reactions are essentially kinetic competitions, the concentration of the rAd would also be expected to influence its final DP. To evaluate this dependence, fluoro-PEG-rAds were prepared at either 0.55 or 0.91×10¹² particles/ml with a range of linker concentrations. As shown in FIG. 4A, at a given linker concentration, the DP decreased by about 10-20% at the higher virus concentration. Therefore with this limited difference in the virus concentration the effect on the DP was significant, although clearly secondary to that of the linker concentration.

pH of the pegylation reaction The fluorescein-PEG-SPA used was a carboxylic acid in a powder form. As described above, reaction pH dropped not only due to aminolysis and hydrolysis but also from the physical dissolution of the solid linker in the reaction solution. Therefore, reaction pH control was more difficult in a pegylation reaction using the fluorescein-labeled linker than using a typical neutral linker. For example, in reactions using fluoro-PEG-SPA with 149 mM Tris, an initial pH of 9.0 dropped to 8.9 with 1% linker but to 8.2 with 10% linker (Table 1A). In order to evaluate the reaction pH effect separately from the linker concentration effect, a set of pegylation reactions were carried out at a higher Tris concentration and at a pH closer to its pKa, 8.1. The precise pH control of these pegylation reactions was not possible, but considerably better, with no pH change at 1% linker and 0.4 pH unit change with 10% linker. The DPs obtained from tighter pH control at lower initial basic pH are compared with those obtained from wider pH control at higher initial reaction pH in FIG. 4B. Pegylation at a reduced pH, 0.3-0.4 units lower, led to a modestly higher DP.

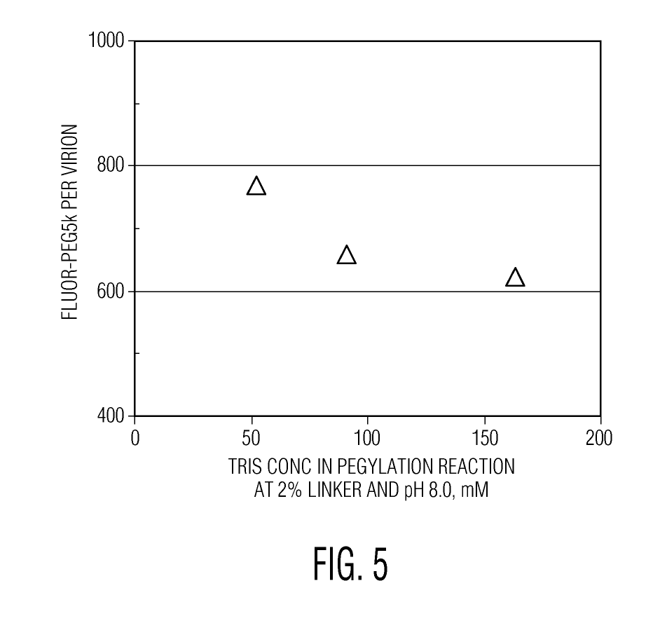

Tris concentration The pH experiments above then focused attention on the potential effect of the Tris concentration on the DP of the rAd. Tris, possessing an amino group, should compete with the amino groups of viral capsid proteins for the linker in the pegylation reaction although the quantification of pegylated Tris is not straightforward. To test effect of Tris concentration a set of pegylation reactions was designed and run at constant virus and linker concentration and relatively constant pH. The DPs of these purified samples were determined by fluorescent SE HPLC (FIG. 5). As the Tris concentration in the pegylation reaction increased from 52 mM to 163 mM, the DP of fluoro-PEG-rAd decreased from 750 to 610 fluorescein-PEG molecules per virion. This result supports the hypothesis that Tris competes with the amino groups of viral proteins in the pegylation reaction and demonstrates the need to give sufficient regard to this variable.

### Example 14

### Stability of Fluoro-PEG-rAds as Evaluated by Fluorescent SE HPLC

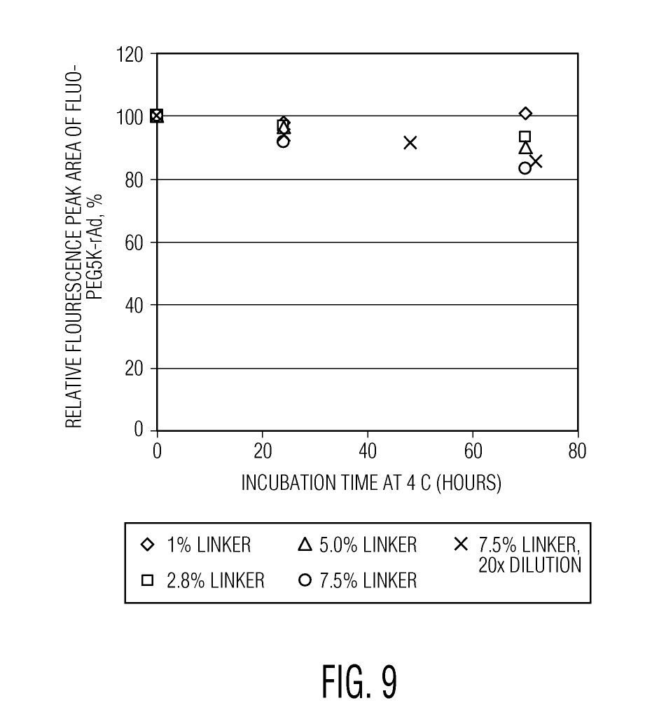

Questions regarding the stability of pegylated rAds were also addressed using the purified fluoro-PEG-rAds. A set of these purified samples produced at differing percent linker concentrations were incubated at 4° C. and periodically analyzed by the fluorescent SE HPLC assay to evaluate any loss of fluorescent PEG that was conjugated to the rAd particles (FIG. 9). There was an apparent dependence on stability relative to the degree of pegylation of the sample: the sample prepared at 1.3% linker concentration showed no change over 72 hours while the sample prepared at 7.4% linker concentration showed an approximate 20 percent reduction in its fluorescence. This loss was not likely an aggregation type of instability since this sample when diluted 20-fold had a loss similar to the undiluted sample.

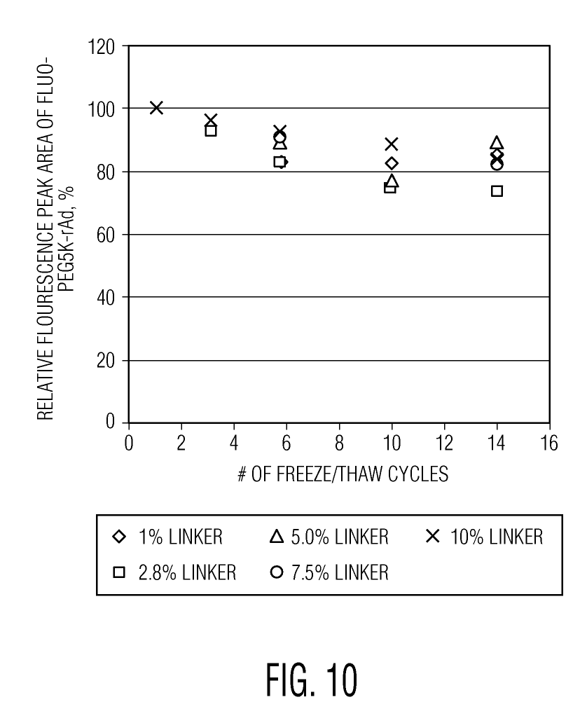

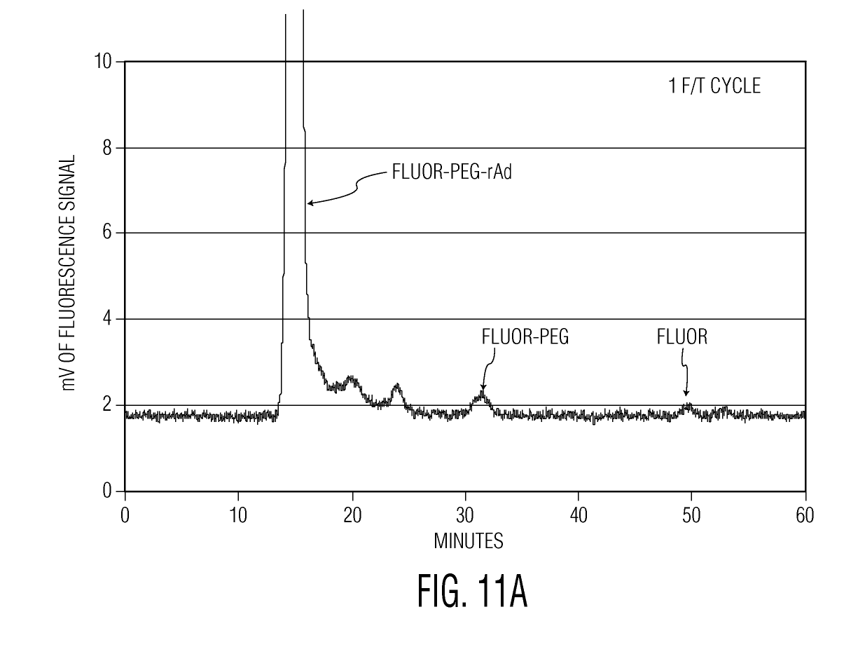

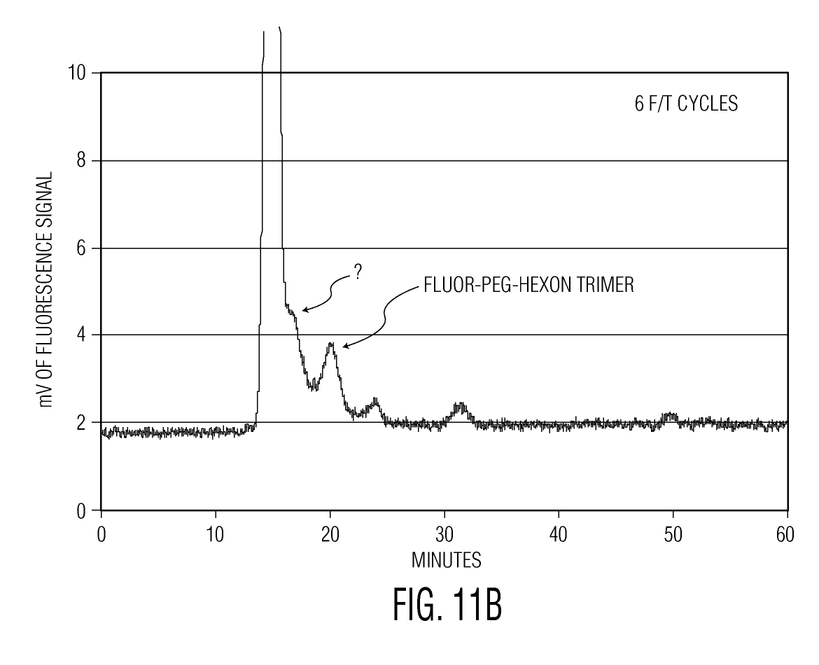

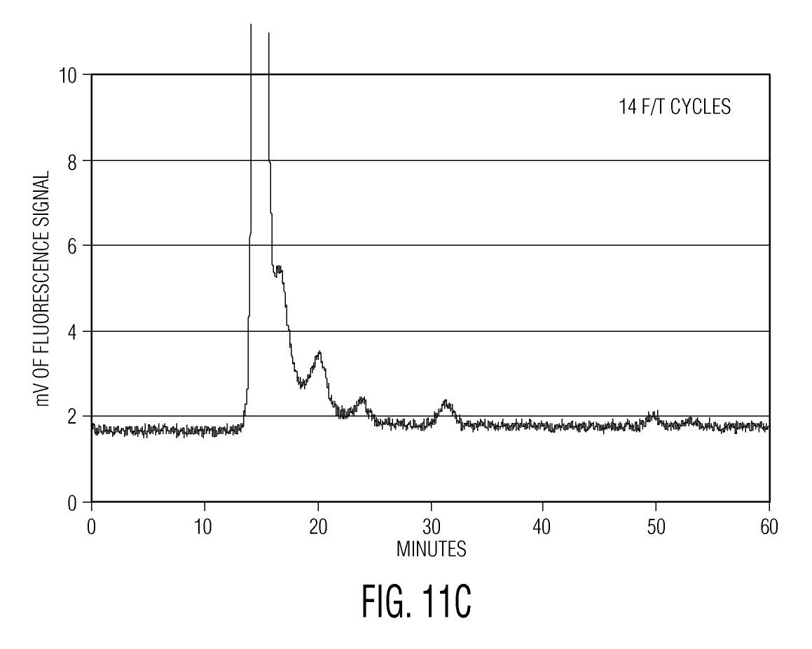

Stability of the set of fluoro-PEG-rAds was also studied after multiple freeze/thaw cycles (FIG. 10). There was some loss (˜20%) in the fluorescence associated with the rAds after 10-14 freeze/thaw cycles but here there was no clear difference between the stability of samples prepared at low or high % linker concentrations. The fluorescent SE HPLC profiles, using a highly expanded scale compared to FIG. 3C, were able to demonstrate subtle changes in the pegylated rAd stability. For example, a fluoro-PEG-rAd sample with 450 DP showed a trailing shoulder of the virus peak followed by an additional partially resolved peak, both of which were more pronounced after six freeze/thaws. These peaks likely represent free pegylated penton and pegylated hexon trimer, based on their elution positions (FIG. 11A, B). Neither of these minor peaks further increased after eight more freeze/thaw cycles. (FIG. 11C). This suggests that there was a minor population of pegylated rAd particles in this sample that was susceptible to disruption by freeze/thaw damage. No increase was seen in the fluoro-PEG or fluorescein peaks indicating that the linkage of the PEG to the rAd was stable.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, Genbank Accession Numbers and protocols are cited throughout this application, the disclosures of which are incorporated herein by reference in their entireties for all purposes.

We claim:

1. A method for determining the relative average degree of polymer attachment of a polymer-particle conjugate preparation having an unknown average degree of polymer attachment, comprising the steps of:
(a) measuring the density of a polymer-particle conjugate preparation having a known average degree of polymer attachment;
(b) measuring the density of a the polymer-particle conjugate preparation having an unknown average degree of polymer attachment; and
(c) comparing the densities of the polymer-particle conjugate preparation having the known average degree of polymer attachment versus the polymer-particle preparation having the unknown average degree of polymer attachment.

2. The method of claim 1, wherein density is measured by analytical ultracentrifugation.

3. The method of claim 1, wherein the polymer is selected from the group consisting of polyethylene glycol, a synthetic polymer, a protein, an oligonucleotide, an oligosaccharide, a lipid and a detergent.

4. The method of claim 3, wherein the polymer is polyethylene glycol.

5. The method of claim 1, wherein the particle comprises a sequence of nucleic acids.

6. The method of claim 5, wherein the particle is a viral vector or an DNA, an RNA or synthetic nucleic acid vector.

7. The method of claim 6, wherein the viral vector is an adenoviral vector.

8. The method of claim 6, wherein the non-viral vector is an oligonucleotide or an oligonucleotide complex.

9. The method of claim 1, wherein the particle is selected from the group consisting of a fullerene, a dendrasome, a nanoparticle, a microparticle and a microgel.

10. The method of claim 1, wherein the polymer-particle conjugate preparation is a pegylated recombinant adenovirus preparation (PEG-rAd), and wherein a lower density indicates a higher degree of pegylation.

11. The method of claim 2, wherein the analytical ultracentrifugation is performed using a density gradient.

12. The method of claim 11, wherein the density gradient is Cesium Chloride, Glycerol, Rubidium Chloride or combinations thereof

13. The method of claim 12, wherein the density gradient is Cesium Chloride.

14. The method of claim 13, wherein the density gradient is a combination of Cesium Chloride and Glycerol.

15. The method of claim 10, wherein the PEG-rAd preparation having the known average degree of pegylation is a fluorescein-labeled PEG-rAd preparation.

16. The method of claim 15, wherein the average degree of pegylation of the fluorescein-labeled PEG-rAd preparation is determined by size exclusion (SE) HPLC with fluorescence quantification of the virus peak.

17. A method for determining the relative average degree of pegylation of a pegylated adenovirus (PEG-rAd) preparation, comprising the steps of:
(a) measuring the density of a PEG-rAd preparation having a known average degree of pegylation;
(b) measuring the density of a PEG-rAd preparation having an unknown average degree of pegylation; and
(c) comparing the densities of the polymer-particle conjugate preparation having the known average degree of polymer attachment versus the polymer-particle preparation having the unknown average degree of polymer attachment; wherein density is measured by analytical ultracentrifugation, and wherein a lower density indicates a higher degree of pegylation.

18. The method of claim 17, wherein the PEG-rAd preparation having the known average degree of pegylation is a fluorescein-labeled PEG-rAd preparation.

19. The method of claim 18, wherein the average degree of pegylation of the fluorescein-labeled PEG-rAd preparation is determined by size exclusion (SE) HPLC with fluorescence quantification of the virus peak.

20. The method of claim 17, wherein the analytical ultracentrifugation is performed on density gradients.

21. The method of claim 20, wherein the density gradient is Cesium Chloride, Glycerol, Rubidium Chloride or combinations thereof.

22. The method of claim 21, wherein the density gradient is Cesium Chloride.

23. The method of claim 21, wherein the density gradient is a combination of Cesium Chloride and Glycerol.

24. The method of claim 21, wherein the density gradient is Glycerol.
